SEQUENCE LISTING (12 sequences)

SEQ ID NO: 1 - DNA, 1779 bp, Homo sapiens
cttctccgctccttctaggatctccgcctggttcggcccgcctgcctccactccagcctctaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagcccccttgtcctggaggtggaccccaacatccaggccgtgcgcacccaggagaaggagcagatcaagaccctcaacaacaagtttgcctccttcatagacaaggtacggttcctggagcagcagaacaagatgctggagaccaagtggagcctcctgcagcagcagaagacggctcgaagcaacatggacaacatgttcgagagctacatcaacaaccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcttacatgaacaaggtagagctggagtctcgcctggaagggctgaccgacgagatcaacttcctcaggcagctatatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctctgagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcgcggcagctgcgtgagtaccaggagctgatgaacgtcaagctggccctggacatcgagatcgccacctacaggaagctgctggagggcgaggagagccggctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactggcaacaggagacccacctgaggctcagccctagccctcagcccacctggggagtttactacctggggaccccccttgcccatgcctccagctacaaaacaattcaattgctttttttttttggtccaaaataaaacctcagctagctctgccaaaaaaaaaaaaaaaaaaaaaaaaaaaa

SEQ ID NO: 2 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 3 - protein, 29 aa, Homo sapiens
MNGVSWSQDLQEGISAWFGPPASTPASTM

SEQ ID NO: 4 - protein, 511 aa, Homo sapiens
MNGVSWSQDLQEGISAWFGPPASTPASTMSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 5 - protein, 16 aa, Drosophila melanogaster
RQIKIWFQNRRMKWKK

SEQ ID NO: 6 - protein, 86 aa, Human immunodeficiency virus
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 7 - protein, 11 aa, Human immunodeficiency virus
YGRKKRRQRRR

SEQ ID NO: 8 - protein, 9 aa, Human immunodeficiency virus
RKKRRQRRR

SEQ ID NO: 9 - protein, 9 aa, artificial sequence
  synthetic peptide
RKKRRQRRR

SEQ ID NO: 10 - protein, 12 aa, artificial sequence
  synthetic peptide
RRQRRTSKLMKR

SEQ ID NO: 11 - protein, 27 aa, artificial sequence
  synthetic peptide
GWTLNSAGYLLGKINLKALAALAKKIL

SEQ ID NO: 12 - protein, 29 aa, artificial sequence
  synthetic peptide
WEAKLAKALAKALAKHLAKALAKALKCEA

(19) United States
(12) Patent Application Publication
(10) Pub. No.: US 2016/0200804 A1
(43) Pub. Date: Jul. 14, 2016

(54) COMPOSITIONS AND METHODS FOR TREATING ANAPLASTIC THYROID CANCER

(71) Applicants: Georgia Regents University, Augusta, GA (US); H. Lee Moffitt Cancer Center and Research Institute, Inc., Tampa, FL (US)

(72) Inventors: Paul Weinberger, Augusta, GA (US); De-Huang Guo, Augusta, GA (US); John Koomen, Tampa, FL (US)

(21) Appl. No.: 14/992,674

(22) Filed: Jan. 11, 2016

Related U.S. Application Data
(60) Provisional application No. 62/101,554, filed on Jan. 9, 2015.

(51) Int. Cl. C07K 16/18; C12N 15/113; A61K 48/00

(52) U.S. Cl. CPC C07K 16/18; A61K 48/00; C12N 15/113; C12Y 301/00; C07K 2317/76; C12N 2310/14; C12N 2320/30

(57) ABSTRACT

Methods of treating a disease or disorder characterized by cells with increased or aberrant expression of cytokeratin-8 (CK8) are disclosed. The methods typically include administering to a subject in need thereof a pharmaceutical composition including an effective amount of a CK8 inhibitor. Exemplary diseases include thyroid cancers, particularly thyroid cancers characterized by poorly differentiated or undifferentiated cells. In the most preferred embodiments the cancer is an anaplastic thyroid cancer or a poorly differentiated papillary thyroid cancer. Disclosed inhibitors include, functional nucleic acids, inhibitory anti-CK8 antibodies, inhibitory peptides, and small molecules.

### CROSS-REFERENCE TO RELATED APPLICATIONS

This application claims the benefit of and priority to U.S. Provisional Application No. 62/101,554, filed Jan. 9, 2015.

### FIELD OF THE INVENTION

The field of the invention is generally related to methods and compositions for treating subjects with diseases and disorders characterized by increased or aberrant expression of cytokeratin-8, such as thyroid cancer.

### BACKGROUND OF THE INVENTION

Anaplastic thyroid cancer (ATC) is a rare form of thyroid carcinoma with extremely high morbidity and fast disease progression. Only 1-2% of thyroid cancers are anaplastic, but the disease contributes to 14-50% of thyroid cancer mortality, and exhibits a median survival of 3 to 5 months (Govardhanan, et al., Journal of Oncology, vol. 2011, Article ID 542358, 13 pages, (2011). All patients with ATC, even those without metastatic disease, are considered to have systemic disease at the time of diagnosis, and all ATCs are considered stage IV by the International Union Against Cancer (UICC)-TNM staging and American Joint Commission on Cancer (AJCC) system. The 5 year overall survival is about 4%.

Standard treatment for ATC typically includes concurrent or sequential chemotherapy and radiation, preferably also in combination with surgery (Govardhanan, et al., Journal of Oncology, vol. 2011, Article ID 542358:1-13, (2011)). The most common cause of death is invasion of local structures in the neck. It is this proximity and invasion of the cancer to vital anatomy that also limits the effectiveness of surgery as a treatment. Doxorubicin is the most commonly used chemotherapeutic agent, and exhibits a response rate of 22%. Radiation when combined with chemotherapy and/or surgery it can increase survival time in some patients, but is not curative. There is an urgent need for more effective compositions and methods of treatment for ATC.

Therefore, it is an object of the invention to provide methods and compositions for treating ATC and other aggressive thyroid cancers.

It is another object of the invention to provide methods and compositions for treating pathologies related to the overexpression of keratins.

### SUMMARY OF THE INVENTION

Methods of treating a disease or disorder characterized by cells with increased or aberrant expression of cytokeratin-8 (CK8) are disclosed. The methods typically include administering to a subject in need thereof a pharmaceutical composition including an effective amount of a CK8 inhibitor. Exemplary diseases include thyroid cancers, particularly thyroid cancers characterized by poorly differentiated or undifferentiated cells. In the most preferred embodiments the cancer is an anaplastic thyroid cancer or a poorly differentiated papillary thyroid cancer.

Suitable inhibitors of CK8 for use in the disclosed methods are also provided. The CK8 inhibitors typically reduce a bioactivity of CK8. For example, the CK8 inhibitor can reduce expression or localization of CK8 protein or mRNA, increases degradation of CK8 protein or mRNA, or a combination thereof. In preferred embodiments, the CK8 inhibitor reduces proliferation of the cells, or a combination thereof.

In some embodiments, the CK8 inhibitor is a functional nucleic acid, or vector encoding the same, selected from the group consisting of antisense oligonucleotides, siRNA, shRNA, miRNA, EGSs, ribozymes, and aptamers. In some embodiments, the functional nucleic acid reduces expression of a nucleic acid with at least 80% sequence identity to the CK8 mRNA sequence encoded by SEQ ID NO:1, or a nucleic acid with at least 80% sequence identity to a polynucleotide encoding the CK8 amino acid sequences of SEQ ID NO:2 or 4.

In some embodiments, the CK8 inhibitor is one or more vectors encoding a gene editing system that when transfected into the cells reduces, prevents, or otherwise disrupts endogenous expression of CK8. Exemplary gene editing systems include CRISPR/Cas, zinc finger nucleases, and transcription activator-life effector nucleases.

In other embodiments, the CK8 inhibitor is an inhibitory anti-CK8 antibody or an antigen-binding fragment thereof. The antibody can be an intact antibody or an antigen binding fragment thereof. The antibody can be an intrabody or a transbody. In some embodiments, delivery of antibodies across the plasma membrane to intracellular CK8 epitopes is facilitated by administering the antibody in a cationic lipid composition.

In some embodiments, the CK8 inhibitor is an inhibitory peptide, for example a peptide that reduces or prevents phosphorylation of or ATP-binding to CK8. In some embodiments, the inhibitory peptide reduces or prevents binding of CK8 to one or more of its binding partners, for example, cytokeratin-18 and or.

In still other embodiments, the CK8 inhibitor is a small molecule. Methods of identifying CK8 inhibitors and for selecting patients for treatment with the disclosed compositions are also provided.

### BRIEF DESCRIPTION OF THE DRAWINGS

FIG. 1 is a bar graph showing the relative cytokeratin-8 expression levels in tall cell variants of papillary thyroid carcinoma cells (Tall Cell PTC), anaplastic thyroid carcinoma cells (ATC), papillary thyroid carcinoma cells (PTC), PTC met cells (PTC met), and control assays including adj. normal cells, stromal cells, and a negative control.

FIG. 2 is a bar graph showing the relative population doubling times (Td) in hours (hrs) of fast growing thyroid cancer cell lines ATC1, 29T, and 11T, and slow growing cell lines 16T, FTC133, and 11T.

FIG. 3 is a homology tree illustrating the results of a BLASTp alignment of cytokeratin-8 peptide fragment SEQ ID NO: to other known proteins.

FIG. 4 is a bar graph showing the results of Phosphotyrosine (pY) Immunoprecipitation (IP)-LC/MS-MS (Normalized Spectrum Count) of fast-growing (from left-to-right, the first six x-axis labels) and slow-growing thyroid cancer cells lines (from left-to-right, the last four x-axis labels).

FIG. 5 is an illustration of a putative signaling pathway modulated by cytokeratin-8.

FIG. 6 is a bar graph of KRT8 expression (log 2(FC) in the indicated solid tumors.

FIG. 7 is a scatter plot of gene expression (thousands) in the indicated tumor type.

FIG. 8 is a bar graph of alteration frequency (%) for the indicated tumor.

FIG. 9 is a scatter plot for keratin-8 expression (nuance score) for benign multinodular goiter (BNG), anaplastic thyroid cancer (ATC) and normal tissue (Norm).

FIG. 10 is a western blot of the indicated cancer cell lines for KRT8 or GAPDH.

FIGS. 11A-11C are flow cytometry plots for shRNA knock down of keratin-8 in cells treated with scramble RNA (FIG. 11A) shRNA-CK8 (FIG. 11B), and no shRNA (no LV). FIG. 11D is a bar graph of apoptotic cells (mean, %) for shRNA CK8 LV treated cells, scramble LV treated cells, and no LV treated cells.

FIGS. 12A and 12B are fluorescence micrographs of cells treated with Tet (KRT8 KD) (FIG. 12B) and a control (FIG. 12A). FIG. 12C is a bar graph of cl-Ca3 protein expression for untreated cells (no Tet) and treated cells (Tet 1.0 ug/ml KRT8 KD).

FIGS. 13A and 13B are bar graphs of keratin-8 expression, % for cells treated with THJ29TPCDNA3.1+ctrl (FIG. 13A) and THJ29TPCDNA3.1+KRT8 (FIG. 13B). FIG. 13C is a bar graph of apoptotic cells (mean, %) for cells treated for 24 or 48 hours with a control (left bar in each set) or with KRT8 (right bar for each set).

FIG. 14A is photograph of a protein separation gel. FIG. 14B is a mass spectrograph of the proteins in FIG. 14A.

FIG. 15 is a western blot probe for KRT8 and ANXA2 where indicated.

FIG. 16A is a bar graph of fold change (%) for ACT^{+tetR+ck8shRNA#c} cells untreated or treated with tetracycline and for ACT cells treated with tetracycline. FIG. 16B is a bar graph of keratin 9 protein expression (%) in wildtype cells, and ACT^{+tetR+ck8shRNA#c} cells treated for 24 or 48 hours respectively.

### DETAILED DESCRIPTION OF THE INVENTION

### I. Definitions

As used herein, “identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and)(BLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, “inhibit” or other forms of the word such as “inhibiting” or “inhibition” means to hinder or restrain a particular characteristic. It is understood that this is typically in relation to some standard or expected value, in other words it is relative, but that it is not always necessary for the standard or relative value to be referred to. For example, “inhibits cytokeratin-8” means hindering or restraining the activity of the protein relative to a standard or a control. “Inhibits cytokeratin-8” can also mean to hinder or restrain the synthesis or expression of the protein relative to a standard or control.

As used herein, “treatment” or “treating” means to administer a composition to a subject or a system with an undesired condition. The condition can include a disease. “Prevention” or “preventing” means to administer a composition to a subject or a system at risk for the condition. The condition can include a predisposition to a disease. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur. It is understood that where treat or prevent are used, unless specifically indicated otherwise, the use of the other word is also expressly disclosed.

As used herein, “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. A subject can include a control subject or a test subject.

As used herein, “operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

As used herein, “localization signal or sequence or domain or ligand” or “targeting signal or sequence or domain or ligand” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location.

As used herein, “microparticles” refers to particles having a diameter between one micron and 1000 microns, typically less than 400 microns, more typically less than 100 microns, most preferably for the uses described herein in the range of less than 10 microns in diameter. Microparticles include microcapsules and microspheres unless otherwise specified.

As used herein, “nanoparticles” refer to particles having a diameter of less than one micron, more typically between 50 and 1000 nanometers, preferably in the range of 100 to 300 nanometers.

As used herein, the “bioactivity” of cytokeratin-8 (CK8) refers to the biological function of the CK8 polypeptide, and most typically relates to its effect on, interaction with, or response from a cell. Bioactivity can be reduced by reducing the availability of CK8 to carry out a biological function of CK8, reducing the activity CK8 polypeptide, reducing the avidity of CK8 polypeptide for one or more of its binding partners, reducing the quantity of CK8 polypeptide, reducing the expression levels of the CK8 mRNA or polypeptide, or a combination thereof. Bioactivity can be increased by increasing the availability of CK8 to carry out a biological function of CK8, increasing the activity CK8 polypeptide, increasing the avidity of CK8 polypeptide for one or more of its binding partners, increasing the quantity of CK8 polypeptide, increasing the expression levels of the CK8 mRNA or polypeptide, or a combination thereof.

### II. Compositions

It has been discovered that in ATC patient samples and patient-derived ATC cell lines, keratin-8 expression correlates with cancer cell growth and tumor progression. The finding that RNA-interference based keratin-8 silencing increases apoptosis and reduces cell viability, while forced overexpression confers resistance to apoptosis under redox stress suggests the possibility that keratin-8 may itself be a driver in ATC tumor biology. This is the first report providing direct evidence that keratin-8 itself is a fundamentally important driver of ATC tumor cell proliferation and survival. The data shows keratin-8 is a novel therapeutic target in ATC. Because there are currently no therapies proven effective for treating this disease, and the 5-year overall survival rate remains at less than 5%, such novel targets are desperately needed. Thus compositions for inhibiting the expression, bioavailability, or biological activity of keratin 8 are provided for treating keratin 8 related cancers such as ATC.

The idea that keratin-8 may play a direct role in ATC tumor biology, while unexpected, is not without precedence. Indeed, many of the other members of the Intermediate Filament family of proteins (which encompasses the keratins) play dual roles as both structural support proteins and intracellular signaling messengers. This concept of “protein moonlighting” among proteins formerly thought to be only structural constituents is an emerging field and provides several models upon which to base future mechanistic studies of keratin-8. For example, beta-catenin, actin, tubulin, and lens crystallins were all at one point thought to be simple structural proteins, and are now known to have significant signaling functions as well.

The data from co-immunoprecipitation experiments suggest reciprocal binding between annexin-A2 and keratin-8. Therefore, some embodiments provide compositions and methods for interfering with the interaction between annexin-A2 and keratin-8 for the treatment of cancer. This is potentially important, as annexin-A2 has known interactions with both the redox and apoptosis pathways. Annexin-A2 has been shown to act in some cancers as a redox sink for peroxide molecules, thus allowing rapid detoxification of peroxide intermediates generated by the elevated metabolic rate in most cancer cells. It is also regulated by reversible glutathionylation, and may mediate free radical and radiation-induced apoptosis. Interestingly, annexin-A2 has also been implicated in gemcitabine resistance in pancreatic cancer, via the AKT/mTOR pathway. One of the defining clinical features of anaplastic thyroid carcinoma is resistance to traditional chemotherapeutic agents. The possibility of keratin-8/annexin-A2 partially mediating this resistance is an intriguing possibility that warrants further investigation.

### A. Cytokerin-8 Inhibitors

Compositions that reduce the bioactivity of cytokeratin-8 (CK8) are provided. The compositions typically include an inhibitor of CK8 that blocks, reduces, or inhibits expression, activity, or availability of CK8.

CK8 (also referred to as Keratin, type II cytoskeletal 8 and K2C8) belongs to the intermediate filament family of proteins. It has been discovered that CK8 expression is increased in aggressive, fast-growing forms of thyroid cancer cells relative to slower growing, less aggressive thyroid cancer cells and normal cells. The Examples below also show that reducing the expression of CK8 in aggressive anaplastic cancer cells overexpressing CK8 protein reduces proliferation of the cells. Without being bound by theory, it is believed that CK8 is a modulator of a signal transduction pathway that ultimately regulates cell proliferation in these cells. Therefore, inhibitors for reducing the bioactivity of CK8 and methods of use thereof for treating diseases and disorder characterized by over- or aberrant expression of CK8 are provided.

In preferred embodiments, the inhibitor reduces the expression CK8 in cells. In some embodiments, the inhibitor additionally or alternatively (1) reduces the binding of ATP to CK8, (2) reduces the phosphorylation of CK8, (3) reduces the availability, expression, and/or activity of upstream or downstream molecules in a CK8 signal transduction pathway that induces or increases target cell proliferation, or (4) a combination thereof. In preferred embodiments, the inhibitor reduces cell proliferation in target cells that are overexpressing or have up-regulated CK8, and/or have aberrant or overactive CK8-based signal transduction. In some embodiments, CK8 phosphorylation is reduced at Ser-23, Ser-73, and/or Ser-431, CK8 binding to CK18 is reduced, or combination thereof. In some embodiments cell-cell adhesion or cell-matrix adhesion is reduced. In the most preferred embodiments, the target cells are over-proliferating or fast-growing cancer cells, such as, but not limited to, anaplastic thyroid cancer cells.

### 1. Functional Nucleic Acids Inhibitors of CK8

The CK8 inhibitor can be a functional nucleic acid. Functional nucleic acids are nucleic acid molecules that have a specific function, such as binding a target molecule or catalyzing a specific reaction. As discussed in more detail below, functional nucleic acid molecules can be divided into the following non-limiting categories: antisense molecules, siRNA, miRNA, aptamers, ribozymes, triplex forming molecules, RNAi, and external guide sequences. The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Therefore the compositions can include one or more functional nucleic acids designed to reduce expression of the gene encoding CK8 (e.g., KRT8), or a gene product thereof.

### a. CK8 Sequences

In some embodiments, the composition includes a functional nucleic acid or polypeptide designed to target and reduce or inhibit expression or translation of CK8 mRNA; or to reduce or inhibit expression, reduce activity, or increase degradation of CK8 protein. In some embodiments, the composition includes a vector suitable for in vivo expression of the functional nucleic acid.

Nucleic acid and amino acid sequences for CK8 are known in the art. See, for example, GenBank Accession No.: BC000654.2 Homo sapiens keratin 8, mRNA (cDNA clone MGC: 1711 IMAGE: 3349233), complete cds which provides the nucleic acid sequence:

| (SEQ ID NO: 1) | |
|---|---|
| 1 | CTTCTCCGCT CCTTCTAGGA TCTCCGCCTG GTTCGGCCCG CCTGCCTCCA CTCCAGCCTC |
| 61 | TACCATGTCC ATCAGGGTGA CCCAGAAGTC CTACAAGGTG TCCACCTCTG GCCCCCGGGC |
| 121 | CTTCAGCAGC CGCTCCTACA CGAGTGGGCC CGGTTCCCGC ATCAGCTCCT CGAGCTTCTC |
| 181 | CCGAGTGGGC AGCAGCAACT TTCGCGGTGG CCTGGGCGGC GGCTATGGTG GGGCCAGCGG |
| 241 | CATGGGAGGC ATCACCGCAG TTACGGTCAA CCAGAGCCTG CTGAGCCCCC TTGTCCTGGA |
| 301 | GGTGGACCCC AACATCCAGG CCGTGCGCAC CCAGGAGAAG GAGCAGATCA AGACCCTCAA |
| 361 | CAACAAGTTT GCCTCCTTCA TAGACAAGGT ACGGTTCCTG GAGCAGCAGA ACAAGATGCT |
| 421 | GGAGACCAAG TGGAGCCTCC TGCAGCAGCA GAAGACGGCT CGAAGCAACA TGGACAACAT |
| 481 | GTTCGAGAGC TACATCAACA ACCTTAGGCG GCAGCTGGAG ACTCTGGGCC AGGAGAAGCT |
| 541 | GAAGCTGGAG GCGGAGCTTG GCAACATGCA GGGGCTGGTG GAGGACTTCA AGAACAAGTA |
| 601 | TGAGGATGAG ATCAATAAGC GTACAGAGAT GGAGAACGAA TTTGTCCTCA TCAAGAAGGA |
| 661 | TGTGGATGAA GCTTACATGA ACAAGGTAGA GCTGGAGTCT CGCCTGGAAG GGCTGACCGA |
| 721 | CGAGATCAAC TTCCTCAGGC AGCTATATGA AGAGGAGATC CGGGAGCTGC AGTCCCAGAT |
| 781 | CTCGGACACA TCTGTGGTGC TGTCCATGGA CAACAGCCGC TCCCTGGACA TGGACAGCAT |
| 841 | CATTGCTGAG GTCAAGGCAC AGTACGAGGA TATTGCCAAC CGCAGCCGGG CTGAGGCTGA |
| 901 | GAGCATGTAC CAGATCAAGT ATGAGGAGCT GCAGAGCCTG GCTGGGAAGC ACGGGGATGA |
| 961 | CCTGCGGCGC ACAAAGACTG AGATCTCTGA GATGAACCGG AACATCAGCC GGCTCCAGGC |
| 1021 | TGAGATTGAG GGCCTCAAAG GCCAGAGGGC TTCCCTGGAG GCCGCCATTG CAGATGCCGA |
| 1081 | GCAGCGTGGA GAGCTGGCCA TTAAGGATGC CAACGCCAAG TTGTCCGAGC TGGAGGCCGC |
| 1141 | CCTGCAGCGG GCCAAGCAGG ACATGGCGCG GCAGCTGCGT GAGTACCAGG AGCTGATGAA |
| 1201 | CGTCAAGCTG GCCCTGGACA TCGAGATCGC CACCTACAGG AAGCTGCTGG AGGGCGAGGA |
| 1261 | GAGCCGGCTG GAGTCTGGGA TGCAGAACAT GAGTATTCAT ACGAAGACCA CCAGCGGCTA |
| 1321 | TGCAGGTGGT CTGAGCTCGG CCTATGGGGG CCTCACAAGC CCCGGCCTCA GCTACAGCCT |
| 1381 | GGGCTCCAGC TTTGGCTCTG GCGCGGGCTC CAGCTCCTTC AGCCGCACCA GCTCCTCCAG |
| 1441 | GGCCGTGGTT GTGAAGAAGA TCGAGACACG TGATGGGAAG CTGGTGTCTG AGTCCTCTGA |
| 1501 | CGTCCTGCCC AAGTGAACAG CTGCGGCAGC CCCTCCCAGC CTACCCCTCC TGCGCTGCCC |
| 1561 | CAGAGCCTGG GAAGGAGGCC GCTATGCAGG GTAGCACTGG CAACAGGAGA CCCACCTGAG |
| 1621 | GCTCAGCCCT AGCCCTCAGC CCACCTGGGG AGTTTACTAC CTGGGGACCC CCCTTGCCCA |
| 1681 | TGCCTCCAGC TACAAAACAA TTCAATTGCT TTTTTTTTTT GGTCCAAAAT AAAACCTCAG |
| 1741 | CTAGCTCTGC CAAAAAAAAA AAAAAAAAAA AAAAAAAAA, |

and the amino acid sequence:

(SEQ ID NO: 2)
MSIRVTQKSY KVSTSGPRAF SSRSYTSGPG SRISSSSFSR
VGSSNFRGGL GGGYGGASGM GGITAVTVNQ SLLSPLVLEV
DPNIQAVRTQ EKEQIKTLNN KFASFIDKVR FLEQQNKMLE
TKWSLLQQQK TARSNMDNMF ESYINNLRRQ LETLGQEKLK
LEAELGNMQG LVEDFKNKYE DEINKRTEME NEFVLIKKDV
DEAYMNKVEL ESRLEGLTDE INFLRQLYEE EIRELQSQIS
DTSVVLSMDN SRSLDMDSII AEVKAQYEDI ANRSRAEAES
MYQIKYEELQ SLAGKHGDDL RRTKTEISEM NRNISRLQAE
IEGLKGQRAS LEAAIADAEQ RGELAIKDAN AKLSELEAAL
QRAKQDMARQ LREYQELMNV KLALDIEIAT YRKLLEGEES
RLESGMQNMS IHTKTTSGYA GGLSSAYGGL TSPGLSYSLG
SSFGSGAGSS SFSRTSSSRA VVVKKIETRD GKLVSESSDV
LPK.

The data below indicates that one or more of amino acid residues 1-10 numbering for the N-terminus of SEQ ID NO:2, may be, or contribute to, an ATP-binding site.

Additional information regarding CK8 sequences is known in the art. See, for example, UniProt Accession No. P05787-K2C8_HUMAN, Nov. 11, 2014, 22 pages, which is specifically incorporated by reference herein in its entirety, and provides sequence variants, and annotates putative function domains and post-translations modifications. For example, SEQ ID NO:2 represents a canonical sequence for CK8 isoform 1. CK8 isoform 2 has amino acid sequence of SEQ ID NO:2, but wherein the methionine at the first position of SEQ ID NO:2 is replaced with

(SEQ ID NO: 3)
MNGVSWSQDL QEGISAWFGP PASTPASTM.

Therefore a canonical amino acid sequence for isoform 2 is

(SEQ ID NO: 4)
MNGVSWSQDL QEGISAWFGP PASTPASTMS IRVTQKSYKV
STSGPRAFSS RSYTSGPGSR ISSSSFSRVG SSNFRGGLGG
GYGGASGMGG ITAVTVNQSL LSPLVLEVDP NIQAVRTQEK
EQIKTLNNKF ASFIDKVRFL EQQNKMLETK WSLLQQQKTA
RSNMDNMFES YINNLRRQLE TLGQEKLKLE AELGNMQGLV
EDFKNKYEDE INKRTEMENE FVLIKKDVDE AYMNKVELES
RLEGLTDEIN FLRQLYEEEI RELQSQISDT SVVLSMDNSR
SLDMDSIIAE VKAQYEDIAN RSRAEAESMY QIKYEELQSL
AGKHGDDLRR TKTEISEMNR NISRLQAEIE GLKGQRASLE
AAIADAEQRG ELAIKDANAK LSELEAALQR AKQDMARQLR
EYQELMNVKL ALDIEIATYR KLLEGEESRL ESGMQNMSIH
TKTTSGYAGG LSSAYGGLTS PGLSYSLGSS FGSGAGSSSF
SRTSSSRAVV VKKIETRDGK LVSESSDVLP K.

In some embodiments, a functional nucleic acid or polypeptide is designed to target a segment of the nucleic acid sequence of SEQ ID NO:1, or the complement thereof, or variants thereof having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1.

In some embodiments, a functional nucleic acid or polypeptide is designed to target a segment of a the nucleic acid encoding the amino acid sequence of SEQ ID NO:2 (isoform 1), SEQ ID NO:4 (isoform 2), or a complement thereof, or a variant thereof having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a nucleic acid encoding the amino acid sequence of SEQ ID NO:2, SEQ ID NO:4 (isoform 2), or the complement thereof.

In some embodiments, the function nucleic acid hybridizes to the nucleic acid of SEQ ID NO:1, or a complement thereof, for example, under stringent conditions. In some embodiments, the functional nucleic acid hybridizes to a nucleic acid sequence that encodes SEQ ID NO:2, SEQ ID NO:4 (isoform 2), or a complement thereof, for example, under stringent conditions.

### b. Functional Nucleic Acids

### i. Antisense

The functional nucleic acids can be antisense molecules. Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. There are numerous methods for optimization of antisense efficiency by finding the most accessible regions of the target molecule. Exemplary methods include in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹².

### ii. Aptamers

The functional nucleic acids can be aptamers. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP and theophiline, as well as large molecules, such as reverse transcriptase and thrombin. Aptamers can bind very tightly with K_{d}'s from the target molecule of less than 10⁻¹²M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is preferred that the aptamer have a K_{d} with the target molecule at least 10, 100, 1000, 10,000, or 100,000 fold lower than the K_{d} with a background binding molecule. It is preferred when doing the comparison for a molecule such as a polypeptide, that the background molecule be a different polypeptide.

### iii. Ribozymes

The functional nucleic acids can be ribozymes. Ribozymes are nucleic acid molecules that are capable of catalyzing a chemical reaction, either intramolecularly or intermolecularly. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes. There are also a number of ribozymes that are not found in natural systems, but which have been engineered to catalyze specific reactions de novo. Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

### iv. Triplex Forming Oligonucleotides

The functional nucleic acids can be triplex forming molecules. Triplex forming functional nucleic acid molecules are molecules that can interact with either double-stranded or single-stranded nucleic acid. When triplex molecules interact with a target region, a structure called a triplex is formed in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹².

### v. External Guide Sequences

The functional nucleic acids can be external guide sequences. External guide sequences (EGSs) are molecules that bind a target nucleic acid molecule forming a complex, which is recognized by RNase P, which then cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukarotic cells. Representative examples of how to make and use EGS molecules to facilitate cleavage of a variety of different target molecules are known in the art.

vi. RNA Interference

In some embodiments, the functional nucleic acids induce gene silencing through RNA interference. Gene expression can also be effectively silenced in a highly specific manner through RNA interference (RNAi). This silencing was originally observed with the addition of double stranded RNA (dsRNA) (Fire, et al. (1998) Nature, 391:806-11; Napoli, et al. (1990) Plant Cell 2:279-89; Hannon, (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, et al. (2001) Genes Dev., 15:188-200; Bernstein, et al. (2001) Nature, 409:363-6; Hammond, et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs.

Sequence specific gene silencing can be achieved in mammalian cells using synthetic, short double-stranded RNAs that mimic the siRNAs produced by the enzyme dicer (Elbashir, et al. (2001) Nature, 411:494 498) (Ui-Tei, et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit.

The production of siRNA from a vector is more commonly done through the transcription of a short hairpin RNAse (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors.

In some embodiment, the functional nucleic acid is siRNA, shRNA, miRNA. In some embodiments, the composition includes a vector expressing the functional nucleic acid. Methods of making and using vectors for in vivo expression of functional nucleic acids such as antisense oligonucleotides, siRNA, shRNA, miRNA, EGSs, ribozymes, and aptamers are known in the art.

### vii. Other Gene Editing Compositions

In some embodiments the functional nucleic acids are gene editing compositions. Gene editing compositions can include nucleic acids that encode an element or elements that induce a single or a double strand break in the target cell's genome, and optionally a polynucleotide. The compositions can be used, for example, to reduce or otherwise modify expression of CK8.

### 1. Strand Break Inducing Elements

CRISPR/Cas

In some embodiments, the element that induces a single or a double strand break in the target cell's genome is a CRISPR/Cas system. CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a Cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the ‘target sequence’ and the tracrRNA is often referred to as the ‘scaffold’.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

Zinc Finger Nucleases

In some embodiments, the element that induces a single or a double strand break in the target cell's genome is a nucleic acid construct or constructs encoding a zinc finger nucleases (ZFNs). ZFNs are typically fusion proteins that include a DNA-binding domain derived from a zinc-finger protein linked to a cleavage domain.

The most common cleavage domain is the Type IIS enzyme Fok1. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992):4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31,978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys₂His₂ zinc fingers, each of which generally recognizes three to four nucleotides in the target DNA sequence. The Cys₂His₂ domain has a general structure: Phe (sometimes Tyr)-Cys-(2 to 4 amino acids)-Cys-(3 amino acids)-Phe(sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

### Transcription Activator-Like Effector Nucleases

In some embodiments, the element that induces a single or a double strand break in the target cell's genome is a nucleic acid construct or constructs encoding a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). US Published Application No. 2011/0145940, which discloses TAL effectors and methods of using them to modify DNA. Miller et al. Nature Biotechnol 29: 143 (2011) reported making TALENs for site-specific nuclease architecture by linking TAL truncation variants to the catalytic domain of Fok1 nuclease. The resulting TALENs were shown to induce gene modification in immortalized human cells. General design principles for TALE binding domains can be found in, for example, WO 2011/072246.

### 2. Gene Altering Polynucleotides

The nuclease activity of the genome editing systems described herein cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site.

Therefore, in some embodiments, the genome editing composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

Accordingly, cleavage of DNA by the genome editing composition can be used to delete nucleic acid material from a target DNA sequence by cleaving the target DNA sequence and allowing the cell to repair the sequence in the absence of an exogenously provided donor polynucleotide. Alternatively, if the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, the compositions can be used to modify DNA in a site-specific, i.e., “targeted”, way, for example gene knock-out, gene knock-in, gene editing, gene tagging, etc. as used in, for example, gene therapy.

In applications in which it is desirable to insert a polynucleotide sequence into a target DNA sequence, a polynucleotide including a donor sequence to be inserted is also provided to the cell. By a “donor sequence” or “donor polynucleotide” or “donor oligonucleotide” it is meant a nucleic acid sequence to be inserted at the cleavage site. The donor polynucleotide typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence flanked by two regions of homology, such that homology-directed repair between the target DNA region and the two flanking sequences results in insertion of the non-homologous sequence at the target region.

### c. Oligonucleotide Composition

The functional nucleic acids can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides comprise uracil, thymine, cytosine, adenine and guanine as the heterocyclic bases, and ribose or deoxyribose sugar linked by phosphodiester bonds.

In some embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In some embodiments, the analogs have a substantially uncharged, phosphorus containing backbone.

### i. Heterocyclic Bases

The principal naturally-occurring nucleotides include uracil, thymine, cytosine, adenine and guanine as the heterocyclic bases. The oligonucleotides can include chemical modifications to their nucleobase constituents. Chemical modifications of heterocyclic bases or heterocyclic base analogs may be effective to increase the binding affinity or stability in binding a target sequence. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-.beta.-D-ribofuranosyl)pyridine (2-aminopyridine), and various pyrrolo- and pyrazolopyrimidine derivatives.

### ii. Sugar Modifications

Oligonucleotides can also contain nucleotides with modified sugar moieties or sugar moiety analogs. Sugar moiety modifications include, but are not limited to, 2′-O-aminoetoxy, 2′-O-amonioethyl (2′-OAE), 2′-O-methoxy, 2′-O-methyl, 2-guanidoethyl (2′-OGE), 2′-0,4′-C-methylene (LNA), 2′-O-(methoxyethyl) (2′-OME) and 2′-O—(N-(methyl)acetamido) (2′-OMA). 2′-O-aminoethyl sugar moiety substitutions are especially preferred because they are protonated at neutral pH and thus suppress the charge repulsion between the TFO and the target duplex. This modification stabilizes the C3′-endo conformation of the ribose or dexyribose and also forms a bridge with the i-1 phosphate in the purine strand of the duplex.

In some embodiments, the functional nucleic acid is a morpholino oligonucleotide. Morpholino oligonucleotides are typically composed of two more morpholino monomers containing purine or pyrimidine base-pairing moieties effective to bind, by base-specific hydrogen bonding, to a base in a polynucleotide, which are linked together by phosphorus-containing linkages, one to three atoms long, joining the morpholino nitrogen of one monomer to the 5′ exocyclic carbon of an adjacent monomer. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337.

Important properties of the morpholino-based subunits typically include: the ability to be linked in a oligomeric form by stable, uncharged backbone linkages; the ability to support a nucleotide base (e.g. adenine, cytosine, guanine, thymidine, uracil or inosine) such that the polymer formed can hybridize with a complementary-base target nucleic acid, including target RNA, with high Tₘ, even with oligomers as short as 10-14 bases; the ability of the oligomer to be actively transported into mammalian cells; and the ability of an oligomer:RNA heteroduplex to resist RNAse degradation.

In some embodiments, oligonucleotides employ morpholino-based subunits bearing base-pairing moieties, joined by uncharged linkages, as described above.

### iii. Internucleotide Linkages

Oligonucleotides connected by an internucleotide bond that refers to a chemical linkage between two nucleoside moieties. Modifications to the phosphate backbone of DNA or RNA oligonucleotides may increase the binding affinity or stability oligonucleotides, or reduce the susceptibility of oligonucleotides nuclease digestion. Cationic modifications, including, but not limited to, diethyl-ethylenediamide (DEED) or dimethyl-aminopropylamine (DMAP) may be especially useful due to decrease electrostatic repulsion between the oligonucleotide and a target. Modifications of the phosphate backbone may also include the substitution of a sulfur atom for one of the non-bridging oxygens in the phosphodiester linkage. This substitution creates a phosphorothioate internucleoside linkage in place of the phosphodiester linkage. Oligonucleotides containing phosphorothioate internucleoside linkages have been shown to be more stable in vivo.

Examples of modified nucleotides with reduced charge include modified internucleotide linkages such as phosphate analogs having achiral and uncharged intersubunit linkages (e.g., Sterchak, E. P. et al., Organic. Chem., 52:4202, (1987)), and uncharged morpholino-based polymers having achiral intersubunit linkages (see, e.g., U.S. Pat. No. 5,034,506), as discussed above. Some internucleotide linkage analogs include morpholidate, acetal, and polyamide-linked heterocycles.

In another embodiment, the oligonucleotides are composed of locked nucleic acids. Locked nucleic acids (LNA) are modified RNA nucleotides (see, for example, Braasch, et al., Chem. Biol., 8(1):1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

In some embodiments, the oligonucleotides are composed of peptide nucleic acids. Peptide nucleic acids (PNAs) are synthetic DNA mimics in which the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds. PNAs maintain spacing of heterocyclic bases that is similar to conventional DNA oligonucleotides, but are achiral and neutrally charged molecules. Peptide nucleic acids are comprised of peptide nucleic acid monomers.

Other backbone modifications include peptide and amino acid variations and modifications. Thus, the backbone constituents of oligonucleotides such as PNA may be peptide linkages, or alternatively, they may be non-peptide peptide linkages. Examples include acetyl caps, amino spacers such as 8-amino-3,6-dioxaoctanoic acid (referred to herein as 0-linkers), amino acids such as lysine are particularly useful if positive charges are desired in the PNA, and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Oligonucleotides optionally include one or more terminal residues or modifications at either or both termini to increase stability, and/or affinity of the oligonucleotide for its target. Commonly used positively charged moieties include the amino acids lysine and arginine, although other positively charged moieties may also be useful. Oligonucleotides may further be modified to be end capped to prevent degradation using a propylamine group. Procedures for 3′ or 5′ capping oligonucleotides are well known in the art.

In some embodiments, the functional nucleic acid can be single stranded or double stranded.

### 2. Anti-CK8 Inhibitory Antibodies

Monoclonal and polyclonal antibodies, and antigen binding fragments thereof, that are reactive with epitopes of CK8 and can reduce the bioactivity of CK8 are also disclosed. Thus, in some embodiments, a compound that reduces the bioactivity of CK8 polypeptide is an antibody that specifically binds CK8 and prevents binding of CK8 to a binding partner thereof, or otherwise carrying out a function of CK8 under physiological conditions.

CK8 is most typically found as an intracellular protein localized in the cytoplasm. However, studies also indicate that the protein can be localized to the cell surface, particular in cancer cells (Godfroid, et al., J Cell Sci 99:595-607 (1991), Gires, Biochem Biophys Res Commun, 328, 1154-1162 (2005), Lui, et al., Neoplasia, 10(11):1275-1284 (2008)). Several potential mechanisms have been proposed for the cell surface expression of CK8 on cancer cell surfaces such as lipid binding followed by translocation to the outer membrane (Asch, et al., Biochim Biophys Acta 1034, 303-308 (1990)), penetration and projection through the plasma membrane as part of a protein complex (Hembrough, et al., J Biol Chem, 271:25684-25691 (1996); Vidrich, et al., Ann N Y Acad Sci, 455, 354-370 (1985)), and noncovalent association, or secondary binding, to the cell membrane after proteolytic release from cells into the extracellular space (Chan, et al., Cancer Res., 46, 6353-6359 (1986), Hembrough, et al., Biochem J, 317:763-769 (1996), Chou, et al., J Cell Sci, 105:433-444 (1993)). Studies also indicate that overproduction of cytokeratins by cancer cells may cause increased cell surface expression when the cytokeratins are not efficiently integrated into intermediate filaments (Ditzel, et al., Proc Natl Acad Sci USA, 94:8110-8115), and that at least colorectal cancer cells may have CK8 degradation pathways that differ from those of normal cells (Nishibori, et al., Cancer Res 56, 2752-2757 (1996)). It is also believed that tumor surface-expressed CK8 may contribute to multi-drug resistance of MCF-7/MX cells by enhancing cell-cell matrix adhesion (Lui, et al., Neoplasia, 10(11):1275-1284 (2008)).

Therefore, in some embodiments, a CK8 inhibitory antibody reduces the bioactivity of CK8 by binding to cell-surface CK8, cytoplasmic CK8, or a combination thereof. In some embodiments, a CK8 inhibitory antibody reduces the bioactivity of CK8 by binding to extracellular CK8, intracellular CK8, transmembrane CK8, or a combination thereof. In some embodiments, a CK8 inhibitory antibody reduces cell-matrix adhesion, cell-cell adhesion, or a combination thereof of a cell, preferably a cancer cell, more preferably a thyroid cancer cell, overly or aberrantly expressing CK8. In some embodiments, a CK8 inhibitory antibody reduces the level of CK8 protein, reduces the level of phosphorylated CK8 protein, reduces the cleavage of CK8 protein, reduces the binding of CK8 to one or more of its binding partners, reduces ATP-binding to CK8, or a combination thereof in a cell, preferably a cancer cell, more preferably a thyroid cancer cell, overly or aberrantly expressing CK8.

Accordingly, both cell membrane penetrating and non-penetrating antibodies, and antigen-binding fragments thereof are disclosed.

### a. Antibodies

The antibodies can be xenogeneic, allogeneic, syngeneic, or modified forms thereof, such as humanized or chimeric antibodies. Antiidiotypic antibodies specific for the idiotype of a specific antibody, are also included. Antibodies that can be used include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

The term “antibody” is meant to include both intact molecules as well as fragments thereof that include the antigen-binding site and are capable of binding to a CK8 epitope. These include, Fab and F(ab′)₂ fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry, 12:1130-1135; Sharon, J. et al. (1976) Biochemistry, 15:1591-1594). These various fragments are produced using conventional techniques such as protease cleavage or chemical cleavage (see, e.g., Rousseaux et al., Meth. Enzymol., 121:663-69 (1986)).

Monoclonal antibodies (mAbs) and methods for their production and use are described in Hartlow, E. et al., Antibodies: A Laboratory Manual, Cold Spring Harbor Laboratory Press, Cold Spring Harbor, N. Y., 1988). Antiidiotypic antibodies are described, for example, in Idiotypes in Biology and Medicine, S Karger Pub. 1990.

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography.

The immunogen may include the complete CK8 polypeptide, or fragments or derivatives thereof. Immunogens include, for example, all or a part of SEQ ID NO:2 or 4. In some embodiments the antibody is specific for CK8 isoform 1, or isoform 2. In some embodiments the antibody or antigen binding fragment is designed to bind an epitope on CK8 isoform 2 that is masked or absent on isoform 1 (e.g., an epitope on SEQ ID NO:3).

Monoclonal antibodies can be produced using conventional hybridoma technology, such as the procedures introduced by Kohler and Milstein, Nature, 256:495-97 (1975), and modifications thereof (see above references). An animal, preferably a mouse is primed by immunization with an immunogen as above to elicit the desired antibody response in the primed animal. B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques. Positive clones are subcloned, e.g., by limiting dilution, and the monoclonal antibodies are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single monoclonal antibody can be harvested by decantation, filtration, or centrifugation.

### b. Antibody Fragments

Also disclosed are fragments of antibodies which have bioactivity. The fragments, whether attached to other sequences or not, can include insertions, deletions, substitutions, or other selected modifications of particular regions or specific amino acids residues, provided the activity of the fragment is not significantly altered or impaired compared to the non-modified antibody or antibody fragment.

Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

The antibodies can be modified to improve their therapeutic potential. For example, in some embodiments, the antibody is conjugated to another antibody specific for a second therapeutic target. For example, the antibody can be a fusion protein containing anti-CK8 scFv and a single chain variable fragment of a monoclonal antibody that specifically binds the second therapeutic target. In other embodiments, the antibody is a bispecific antibody having a first heavy chain and a first light chain from an anti-CK8 antibody and a second heavy chain and a second light chain from a monoclonal antibody that specifically binds the second therapeutic target.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

The therapeutic function of the antibody can be enhanced by coupling the antibody or a fragment thereof with a therapeutic agent. Such coupling of the antibody or fragment with the therapeutic agent can be achieved by making an immunoconjugate or by making a fusion protein, or by linking the antibody or fragment to a nucleic acid such as an inhibitory nucleic acid or to a small molecule.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. The DNA sequence will then be expressed by a cell as a single protein. The protein can be engineered to include the full sequence of both original proteins, or only a portion of either. If the two entities are proteins, often linker (or “spacer”) peptides are also added which make it more likely that the proteins fold independently and behave as expected.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

In some embodiments, the antibody is modified to alter its half-life. In some embodiments, it is desirable to increase the half-life of the antibody so that it is present in the circulation or at the site of treatment for longer periods of time. For example, where the antibodies are being used alone to treat cancer, e.g., cancer cells having impaired DNA repair, it may be desirable to maintain titers of the antibody in the circulation or in the location to be treated for extended periods of time. In other embodiments, the half-life of the antibody is decreased to reduce potential side effects. Antibody fragments, such as an scFv, are expected to have a shorter half-life than full size antibodies. Other methods of altering half-life are known and can be used in the described methods. For example, antibodies can be engineered with Fc variants that extend half-life, e.g., using Xtend™ antibody half-life prolongation technology (Xencor, Monrovia, Calif.).

### c. Intracellular Antibodies

As discussed above, in some embodiments, the antibody targets intracellular CK8. Accordingly, systems for intracellular delivery of antibodies and cell-penetrating antibodies are also provided. In some embodiments, the antibody is an intrabody. Intrabodies are genetically-engineered antibody molecules that are ectopically expressed within cells. Intrabodies can directly inhibit the function of the targeted antigen, such as a CK8 antigen, or by diverting the targeted antigen from its normal intracellular location (e.g., an intrabody can redirect its target antigen to the degradation machinery). Intrabodies can also enhance or change the function of their target antigens. For protein targets, intrabodies can be targeted to a specific post-translational modification or to a specific antigen conformation. Moreover, an intrabody-induced inhibition can be confined to a specific cell compartment by targeting an intrabody to the specific subcellular compartment using an addressing signal (e.g., a nuclear localization signal, a mitochondrial localization signal, or an endoplasmic reticulum retention signal). Intrabodies can also modulate target function by modifying the oligomeric structure of the target. Method of making, selecting, and using intrabodies are known in the art (e.g., U.S. Published Application No. 2009/0143247).

An intrabody can be administered to a cell by administering to the cell an expression vector encoding the intrabody of interest. Expression vectors that are suitable for expression of intrabodies are well-known in the art. Administration of expression vectors that encode the intrabody, can be achieved by any one of numerous, well-known approaches, for example, but not limited to, direct transfer of the nucleic acids, in a plasmid or viral expression vector, alone or in combination with carriers such as cationic liposomes, another nucleic acid delivery methods discussed in more detail below. Such expression vectors (which contain promoter and enhancer sequences suitable for expressing an operably-linked coding sequence when the expression vector is introduced into a cell) and methods for making, using, and delivering such vectors to cells are well known in the art and readily adaptable for use for administering intrabodies to cells.

In some embodiments, the antibody is a cell-permeable intrabody (also referred to as a transbody) that is prepared by fusing an scFv antibody with a protein transduction domain (PTD). Suitable PTD are known in the art and discussed in more detail below. Methods of making transbodies are known in the art, see for example, Heng, and Cao, Med Hypotheses 64:1105-1108 (2005), Poungpair, et al., Bioconjug. Chem., 21(7):1134-41 (2010)). When contacted with the cell, transbodies cross the cell membrane and enter the cell where they can bind to intracellular epitopes.

Additionally or alternatively, antibodies, including intact antibodies and antigen binding fragments thereof, can be delivered into cells by utilizing a delivery vehicle such as cationic lipids (Court, et al., Mol. Cancer Ther., 6:1728-36 (2007)).

### 3. Inhibitory Peptides

Inhibitory peptides are also provided. Exemplary peptides include those that bind to CK8 or a binding partner thereof and reduce or inhibit an activity of CK8. In some embodiments, the inhibitory peptide binds to CK8 and reduces or prevents one or more of its activities. In some embodiments, the inhibitory peptide sequesters CK8 in subcellular location that reduces its availability to bind to one or more binding partners. In some embodiments, the inhibitory peptides targets CK8 for degradation or otherwise increases degradation of CK8.

Inhibitory peptides include fragments or variants of full-length CK8 that can bind to CK8 binding partners but have reduced CK8 activity or a reduced ability to be activated (e.g., CK8 mimics). For example, in some embodiments, one or more residues of the inhibitory peptide are substituted or deleted relative to wildtype CK8, reducing the ability of the inhibitory peptide to bind ATP or to be phosphorylated at one or more residues relative to full-length wildtype CK8 (e.g., SEQ ID NO:2 or 4). In some embodiments, the inhibitory peptide serves as a molecular sink or otherwise reduces or shunts signal transduction away for a pro-proliferative signaling cascade.

Phosphorylation of serine residues of CK8 is enhanced during EGF stimulation and mitosis. For example, it is believed that Ser-23, Ser-73, and Ser-431 are major phosphorylation sites of human CK3, and at least phosphorylation at Ser-431 is increasing during proliferation (Ku and Omary, et al., JBC, 272:7556-564 (1997)). Ser-23, Ser-73, and Ser-431 refer to the amino positions numbering for the N-terminus of human CK8 without the N-terminal methionine. Therefore, Ser-23, Ser-73, and Ser-431 as discussed in the art correspond to Ser-24, Ser-74, and Ser-432 of SEQ ID NO:2 (or the corresponding serines in SEQ ID NO:4), which includes the N-terminal methionine. Phosphorylation at Ser-431 increases dramatically upon stimulation of cells with epidermal growth factor (EGF) or after mitotic arrest and is the major CK8 phosphorylated residue after incubating K8 immunoprecipitates with mitogen-activated protein or cdc2 kinases (Ku and Omary, et al., JBC, 272:7556-564 (1997)). Therefore, in some embodiments, the inhibitory peptide reduces or prevents phosphorylation of Ser-24, Ser-74, Ser-432, or a combination thereof of SEQ ID NO:2, or of one or more of the corresponding serines in SEQ ID NO:4.

### 4. Small Molecule Inhibitors of CK8

In some embodiments the CK8 inhibitor is a small molecule. The term “small molecule” generally refers to small organic compounds having a molecular weight of more than about 100 and less than about 2,500 Daltons, preferably between 100 and 2000, more preferably between about 100 and about 1250, more preferably between about 100 and about 1000, more preferably between about 100 and about 750, more preferably between about 200 and about 500 Daltons. The small molecules can include cyclical carbon or heterocyclic structures and/or aromatic or polyaromatic structures substituted with one or more functional groups. Small molecule CK8 inhibitors typically reduce or interfere with one or more bioactivities of CK8 discussed herein or otherwise known in the art.

Modulators of the function, expression, or bioactivity of CK8 including small molecules, inhibitory antibodies, inhibitory nucleic acids, and others can be identified using well known techniques and reagents. In some embodiments, the modulator increases or decreases the physical interaction between CK8 and one or more of its binding partners.

In some embodiments, screening assays include random screening of large libraries of test compounds. Alternatively, the assays may be used to focus on particular classes of compounds suspected of modulating the function or expression of CK8 in cells, tissues, organs, or systems.

Assays can include determinations of protein expression, protein activity, or binding activity of CK8. Other assays include determinations of nucleic acid transcription or translation, for example mRNA levels, miRNA levels, mRNA stability, mRNA degradation, transcription rates, and translation rates of CK8.

In some embodiments, the identification of a CK8 modulator is based on the function of wildtype CK8 in the presence and absence of a test compound. The test compound or modulator can be any substance that alters or is believed to alter the function of CK8. In some embodiments the test compound or modulator increases or decreases the ability of CK8 to bind to a binding partner. In some embodiments the test compound or modulator reduces expression of CK8 and/or reduces an activity of CK8 compared to a control. In some embodiments the test compound or modulator reduces cell-cell or cell-matrix adhesion, or proliferation of a target cell, such as a cancer cell.

One exemplary method includes contacting CK8 with at least a first test compound, and assaying for an interaction between CK8 and the first test compound with an assay.

Specific assay endpoints or interactions that may be measured in the disclosed embodiments include, for example, endogenous CK8 expression levels, cell adhesion, and cell proliferation. These assay endpoints may be assayed using standard methods such as FACS, FACE, ELISA, Northern blotting and/or Western blotting. Moreover, the assays can be conducted in cell free systems, in isolated cells, genetically engineered cells, immortalized cells, or in organisms such as C. elegans and transgenic animals.

Other screening methods include labeling CK8 to identify a test compound. CK8 can be labeled using standard labeling procedures that are well known and used in the art. Such labels include, but are not limited to, radioactive, fluorescent, biological and enzymatic tags.

Some embodiments include a method for identifying a modulator of expression CK8 by determining the effect a test compound has on the expression of endogenous CK8 in cells. For example isolated cells or whole organisms or specific cells or tissue in vivo that are expressing CK8 can be contacted with a test compound. Expression of CK8 can be determined by detecting CK8 protein expression or CK8 mRNA transcription or translation. Suitable cells for this assay include, but are not limited to, immortalized cell lines, primary cell culture, or cells engineered to express CK8. Compounds that modulate the expression of CK8, particularly those that reduce expression of CK8, can be selected.

One example of a cell free assay is a binding assay. While not directly addressing function, the ability of a modulator to bind to a target molecule, for example CK8, or a binding partner thereof, is strong evidence of a related biological effect. The binding of a molecule to a target may, in and of itself, be inhibitory, due to steric, allosteric or charge-charge interactions or may downregulate or inactivate CK8. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

Techniques for high throughput screening of compounds are known in the art. Large numbers of small peptide test compounds can be synthesized on a solid substrate, such as plastic pins or some other surface. Bound polypeptide can be detected by various methods.

### B. Targeting Signal or Domain

The compositions can be optionally modified to include one or more targeting signals, ligands, or domains. The targeting signal can be operably linked with the CK8 inhibitor, or a delivery vehicle such as a microparticle. For example, in some embodiments, the targeting signal is linked or conjugated directly or indirectly to the CK8 inhibitor. In some embodiments, the targeting signal is linked, conjugated, or associated directly, or indirectly, with a delivery vehicle such as a liposome or a nanoparticle. Delivery vehicles are discussed in more detail below. The targeting signal or sequence can be specific for a host, tissue, organ, cell, organelle, non-nuclear organelle, or cellular compartment.

In some embodiments, the targeting signal binds to its ligand or receptor which is located on the surface of a target cell such as to bring the composition or a delivery vehicle thereof and cell membranes sufficiently close to each other to allow penetration of the composition or delivery vehicle into the cell. In a preferred embodiment, the targeting molecule is selected from the group consisting of an antibody or antigen binding fragment thereof, an antibody domain, an antigen, a cell surface receptor, a cell surface adhesion molecule, a major histocompatibility locus protein, a viral envelope protein and a peptide selected by phage display that binds specifically to a defined cell.

Targeting the compositions or delivery vehicles to specific cells can be accomplished by modifying the disclosed compositions or delivery vehicles to express specific cell and tissue targeting signals. These sequences target specific cells and tissues, but in some embodiments the interaction of the targeting signal with the cell does not occur through a traditional receptor:ligand interaction. Eukaryotic cells have a number of distinct cell surface molecules. The structure and function of each molecule can be specific to the origin, expression, character and structure of the cell. Determining the unique cell surface complement of molecules of a specific cell type can be determined using techniques well known in the art.

One skilled in the art will appreciate that the tropism of the compositions or delivery vehicles described can be altered by merely changing the targeting signal. In one specific embodiment, compositions are provided that enable the addition of cell surface antigen specific antibodies to the composition or delivery vehicle for targeting the delivery the CK8 inhibitor to the target cells.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used a targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest.

In some embodiments, the targeting domains bind to antigens, ligands or receptors that are specific to tumor cells or tumor-associated neovasculature, or are upregulated in tumor cells or tumor-associated neovasculature compared to normal tissue.

In some embodiments, the targeting domain includes a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In another embodiment, the fusion proteins contain a domain that specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

In preferred embodiments, the targeting signal or domain targets the CK8 inhibitor or a delivery vehicle carrying the inhibitor to cancer cells, preferably thyroid cancer cells, more preferably poorly differentiated or undifferentiated thyroid cancer cells, most preferably anaplastic thyroid cancer cells.

In some embodiments, the targeting signal is incorporated into or linked to a delivery vehicle. For example, if the delivery vehicle is a polymeric particle, the targeting molecules can be coupled directly to the particle or to an adaptor element such as a fatty acid which is incorporated into the polymer. Ligands may be directly attached to the surface of polymeric particles via a functional chemical group (carboxylic acids, aldehydes, amines, sulfhydryls and hydroxyls) present on the surface of the particle and present on the ligand to be attached. Functionality may be introduced post-particle preparation, by direct crosslinking of particles and ligands with homo- or heterobifunctional crosslinkers. This procedure may use a suitable chemistry and a class of crosslinkers (CDT, EDAC, glutaraldehydes, etc. as discussed in more detail below) or any other crosslinker that couples ligands to the particle surface via chemical modification of the particle surface after preparation.

Ligands may also be attached to polymeric particles indirectly though adaptor elements which interact with the polymeric particle. Adaptor elements may be attached to polymeric particles in at least two ways. The first is during the preparation of the micro- and nanoparticles, for example, by incorporation of stabilizers with functional chemical groups during emulsion preparation of microparticles. For example, adaptor elements, such as fatty acids, hydrophobic or amphiphilic peptides and polypeptides can be inserted into the particles during emulsion preparation. In a second embodiment, adaptor elements may be amphiphilic molecules such as fatty acids or lipids which may be passively adsorbed and adhered to the particle surface, thereby introducing functional end groups for tethering to ligands. Adaptor elements may associate with micro- and nanoparticles through a variety of interactions including, but not limited to, hydrophobic interactions, electrostatic interactions and covalent coupling.

In some embodiments, the targeting signal is or includes a protein transduction domain, also known as cell penetrating peptides (CPPS). PTDs are known in the art, and include but are not limited to small regions of proteins that are able to cross a cell membrane in a receptor-independent mechanism (Kabouridis, P., Trends in Biotechnology (11):498-503 (2003)). The two most commonly employed PTDs are derived from TAT (Frankel and Pabo, Cell, December 23; 55(6):1189-93 (1988)) protein of HIV and Antennapedia transcription factor from Drosophila, whose PTD is known as Penetratin (Derossi et al., J Biol Chem. 269(14):10444-50 (1994)).

The Antennapedia homeodomain is 68 amino acid residues long and contains four alpha helices. Penetratin is an active domain of this protein which consists of a 16 amino acid sequence derived from the third helix of Antennapedia (SEQ ID NO:5). TAT protein (SEQ ID NO:6) consists of 86 amino acids and is involved in the replication of HIV-1. The TAT PTD consists of an 11 amino acid sequence domain (residues 47 to 57; YGRKKRRQRRR (SEQ ID NO:7) of the parent protein that appears to be critical for uptake. Additionally, the basic domain Tat(49-57) or RKKRRQRRR (SEQ ID NO:8) has been shown to be a PTD.

Several modifications to TAT, including substitutions of Glutatmine to Alanine, i.e., Q→A, have demonstrated an increase in cellular uptake anywhere from 90% to up to 33 fold in mammalian cells. (Ho et al., Cancer Res. 61(2):474-7 (2001)) The most efficient uptake of modified proteins was revealed by mutagenesis experiments of TAT-PTD, showing that an 11 arginine stretch was several orders of magnitude more efficient as an intercellular delivery vehicle. Thus, some embodiments include PTDs that are cationic or amphipathic. Additionally exemplary PTDs include but are not limited to poly-Arg-RRRRRRR (SEQ ID NO:9); PTD-5-RRQRRTSKLMKR (SEQ ID NO:10); Transportan GWTLNSAGYLLGKINLKALAALAKKIL (SEQ ID NO:11); KALA-WEAKLAKALAKALAKHLAKALAKALKCEA (SEQ ID NO:12); and RQIKIWFQNRRMKWKK (SEQ ID NO:13).

### C. Delivery Vehicles

The CK8 inhibitors can be administered and taken up into the cells of a subject with or without the aid of a delivery vehicle. Appropriate delivery vehicles for the disclosed inhibitors are known in the art and can be selected to suit the particular inhibitor. For example, if the CK8 inhibitor is a nucleic acid or vector, the delivery vehicle can be a viral vector, for example a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome. The recombinant retrovirus can then be used to infect and thereby deliver to the infected cells nucleic acid encoding the CK8 inhibitor. The exact method of introducing the altered nucleic acid into mammalian cells is, of course, not limited to the use of retroviral vectors. Other techniques are widely available for this procedure including the use of adenoviral vectors, adeno-associated viral (AAV) vectors, lentiviral vectors, pseudotyped retroviral vectors, and others described in (Soofiyani, et al., Advanced Pharmaceutical Bulletin, 3(2):249-255 (2013), which is specifically incorporated by reference herein in its entirety. Viruses can be modified to enhance safety, increase specific uptake, and improve efficiency (see, for example, Zhang, et al., Chinese J Cancer Res., 30(3):182-8 (2011), Miller, et al., FASEB J, 9(2):190-9 (1995), Verma, et al., Annu Rev Biochem., 74:711-38 (2005)).

Physical transduction techniques can also be used, such as liposome delivery and receptor-mediated and other endocytosis mechanisms (see, for example, Schwartzenberger et al., Blood 87:472-478 (1996)). For example in some embodiments, the CK8 inhibitor is delivered via a liposome. Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, the disclosed nucleic acid or vector can be delivered in vivo by electroporation as well as by means of a sonoporation. During electroporation electric pulses are applied across the cell membrane to create a transmembrane potential difference, allowing transient membrane permeation and transfection of nucleic acids through the destabilized membrane (Soofiyani, et al., Advanced Pharmaceutical Bulletin, 3(2):249-255 (2013)). Sonoporation combines the local application of ultrasound waves and the intravascular or intratissue administration of gas microbubbles to transiently increase the permeability of vessels and tissues (Escoffre, et al., Curr Gene Ther., 13(1):2-14 (2013)). Electroporation and ultrasound based techniques are targeted transfection methods because the electric pulse or ultrasound waves can be focused on a target tissue or organ and hence gene delivery and expression should be limited to thereto. The disclosed compositions and methods can be used in conjunction with any of these or other commonly used gene transfer methods, including, but not limited to hydrodynamic injection, use of a gene gun.

In some embodiments, the delivery vehicle is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the CK8 inhibitor. In some embodiments, release of the drug(s) is controlled by diffusion of the CK8 inhibitor out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. Preferred polymeric particles for delivery of nucleic acids are known in the art. See, for example, Nimesh, et al., J Biomed Nanotechnol 7(4):504-20 (2011).

The CK8 inhibitor can be incorporated into prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, waxlike substances and mixtures thereof. Suitable fats and fatty substances include fatty alcohols (such as lauryl, myristyl stearyl, cetyl or cetostearyl alcohol), fatty acids and derivatives, including, but not limited to, fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes.

Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

### III. Methods of Treatment

Methods of using the disclosed compositions are also provided. The methods typically include contacting a pharmaceutical composition including a CK8 inhibitor with a target cell in an effective amount to reduce one or more bioactivities of CK8 in the target cell. Target cells are typically cells that are over- or aberrantly expressing CK8 compared to a control. Preferred bioactivities of CK8 are discussed above. In the most preferred embodiments target cells are contacted with the CK8 inhibitor in vivo by administrating a pharmaceutical composition containing an effective amount of CK8 inhibitor to a subject in need thereof. Most typically the subject has a disease or disorder caused or characterized by target cells with increased or aberrant expression or activity of CK8. The administration can be systemic or can be locally to the target cells, tissue, or organ.

### A. Formulations and Methods of Delivery

Pharmaceutical compositions including one or more CK8 inhibitors, and methods of administration are provided.

### 1. Pharmaceutical Compositions

Pharmaceutical compositions including a CK8 inhibitor, and optionally a targeting moiety, a delivery vehicle, or a combination thereof are provided. Pharmaceutical compositions can be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In certain embodiments, the compositions are administered locally, for example by injection directly into a site to be treated. In some embodiments, the compositions are injected, topically applied, or otherwise administered directly into the vasculature onto vascular tissue at or adjacent to a site of injury, surgery, or implantation. Typically, local administration causes an increased localized concentration of the compositions which is greater than that which can be achieved by systemic administration.

### a. Formulations for Parenteral Administration

Compositions including those containing a CK8 inhibitor, and optionally a targeting moiety, a delivery vehicle, or a combination thereof are administered in an aqueous solution, by parenteral injection. The formulation may also be in the form of a suspension or emulsion. In general, pharmaceutical compositions are provided including effective amounts of the CK8 inhibitor and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### b. Oral Formulations

Oral formulations may be in the form of chewing gum, gel strips, tablets or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations. The components of aerosol formulations include solubilized active ingredients, antioxidants, solvent blends and propellants for solution formulations, and micronized and suspended active ingredients, dispersing agents and propellants for suspension formulations. The oral, aerosol and nasal formulations of the invention can be distinguished from injectable preparations of the prior art because such formulations may be nonaseptic, whereas injectable preparations must be aseptic.

### c. Formulations for Topical Administration

The CK8 inhibitor, and optionally a targeting moiety, a delivery vehicle, or a combination thereof can be applied topically. Topical administration can include application to the lungs, nasal, oral (sublingual, buccal), vaginal, rectal mucosa, and skin.

Compositions can be delivered to the lungs while inhaling and traverse across the lung epithelial lining to the blood stream when delivered either as an aerosol or spray dried particles having an aerodynamic diameter of less than about 5 microns.

A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator.

Transdermal formulations may also be prepared. These will typically be ointments, lotions, sprays, or patches, all of which can be prepared using standard technology. Transdermal formulations can include penetration enhancers.

### 2. Effective Amounts

In some in vivo approaches, the compositions are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect.

The amount of composition administered to the subject is typically effective to reduce or prevent one or more symptoms of a disease to be treated, for example, a cancer such as anaplastic thyroid cancer. In preferred embodiments, the inhibitor is administered in an effective amount to reduce the expression CK8 in cells. In some embodiments, the inhibitor additionally or alternatively administered in an effective amount to (1) reduce the binding of ATP to CK8, (2) reduce the phosphorylation of CK8, (3) reduce the bioactivity, expression, and/or phosphorylation of upstream or downstream molecules in a CK8 signal transduction pathway that induces or increases cell proliferation, or (4) a combination thereof. In preferred embodiments, the inhibitor is administered in an effective amount to reduce cell proliferation in cells that are overexpressing or have up-regulated CK8, and/or have aberrant or overactive CK8-based signal transduction. In the most preferred embodiments, the cells are over proliferating or fast growing cancer cells, such as, but not limited to, anaplastic thyroid cancer cells.

The precise dosage will vary according to a variety of factors such as subject-dependent variables (e.g., age, immune system health, etc.), the disease, and the treatment being effected. As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 50 mg/kg of body weight daily are administered to mammals. Generally, for intravenous injection or infusion, dosage may be lower.

### B. Diseases to Treat

### 1. Thyroid Cancer

Subjects in need of the disclosed compositions typically have a disease or disorder characterized by over- or aberrant expression of CK8. In some embodiments, the subject has cancer. In a more particular embodiment, the subject has a thyroid cancer. Thyroid cancers and the cytological and pathological characteristics thereof are described in Patel, Cancer Control, 13(2):119-128 (2006) which is specifically incorporated by reference herein in its entirety. Thyroid cancers include well-differentiated (WDTC) and differentiated (DTC) forms of papillary and follicular thyroid cancer, and combinations thereof. Differentiated forms of thyroid cancer make up about 90% of all incidents of thyroid cancer, and are characterized by differentiated cells that are almost nearly normal in appearance. There is a high survival rate among subjects with differentiated disease.

Thyroid cancers also include sub-variants and poorly differentiated forms such as Columnar, Tall Cell, Insular, Diffuse Sclerosis, and Hurthle Cell carcinoma (also known as Oxyphil Cell Carcinoma) Such cancers make up about 2-4% of thyroid cancer incidents and characterized by poorly differentiated cells that tend to grow and spread more quickly than differentiated cancer thyroid cancer cells. Poorly differentiated thyroid carcinoma (PDTC), may represent intermediate entities in the progression of WDTC to ATC (Patel, Cancer Control, 13(2):119-128 (2006).

Thyroid cancers also include anaplastic carcinoma (ATC). ATC cells are undifferentiated. It is believed that ATC develops from an existing follicular cancer that further mutated, and can develop from long existing tumors that were left untreated and abruptly became aggressive. ATC makes up about 1.5% of thyroid cancer incidents. It spreads rapidly and is difficult to treat. The prognosis for subjects with ATC is poor.

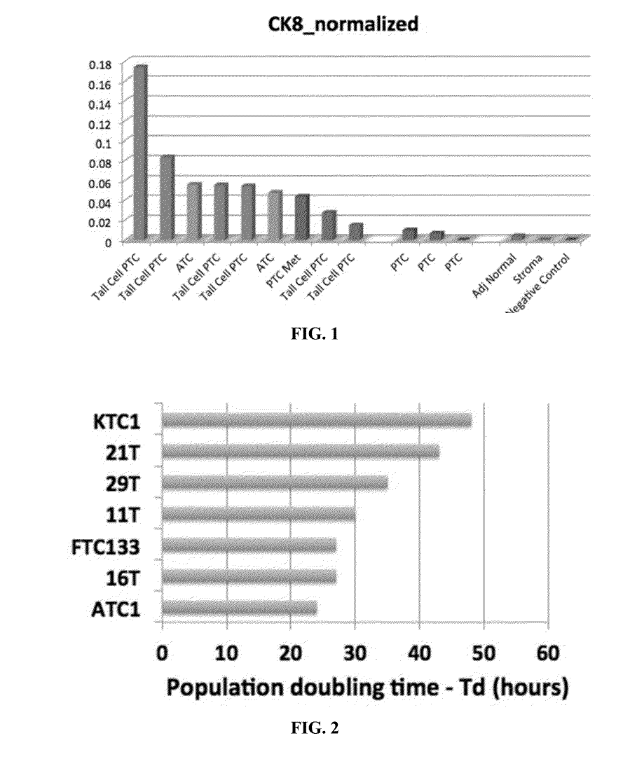

The Examples below illustrate that CK8 is overexpressed in anaplastic thyroid cancer cells relative to normal cells and some less aggressive forms of thyroid cancer. FIG. 1 shows that generally, relative to negative control, CK8 is increasingly expressed in papillary thyroid cancer cells, metastatic papillary thyroid cancer cells, and anaplastic thyroid cancer cells. The expression of CK8 in Tall Cell papillary thyroid cancer cells appears to cell-line dependent can range from less than metastatic papillary thyroid cancer cells to more than anaplastic thyroid cancer cells.

Therefore, in some embodiments, the subject has a differentiated or well-differentiated thyroid cancer. In preferred embodiments, the subject has a poorly differentiated or undifferentiated thyroid cancer. In some embodiments, the cancer is papillary, follicular, papillary-follicular, columnar, tall cell, insular, diffuse sclerosis, Wirthle cell, or anaplastic carcinoma. In preferred embodiments, the subject has a tall cell or anaplastic carcinoma thyroid cancer.

### 2. Other Cancers and Other Diseases to be Treated

In some embodiments, the subject has a cancer characterized by increased or aberrant expression of CK8, but is not a thyroid cancer.

### C. Combination Therapies

The CK8 inhibitors disclosed herein can be administered alone or in combination with each other, or other therapeutic agents. In some embodiments, two therapeutic agents are administered separately, but simultaneously. The two therapeutic agents can also be administered as part of the same admixture. In other embodiments, two therapeutic agents are administered separately and at different times, but as part of the same treatment regime.

In some embodiments the CK8 inhibitor is administered every 1, 2, 3, 4, 5, 6, 7, 8, 9, 10 or more days or weeks. A treatment regime can include 1, 2, 3, 4, 5, 6, 7, 8, 9, 10, 12, 15, 18, 20, 25, 30, 50, 75 or more administrations of the CK8 inhibitor. In some embodiments, a second therapeutic agent is administered every 1, 2, 3, 4, 5, 6, 7, 8, 9, 10 or more days or weeks. A treatment regime can include 1, 2, 3, 4, 5, 6, 7, 8, 9, 10, 12, 15, 18, 20, 25, 30, 50, 75 or more administrations of the second therapeutic agent. The regimen can include administering the CK8 inhibitor and second therapeutic 0, 1, 2, 3, 4, 5, 6, 7, 8, 9, 10 or more days or weeks apart. In some embodiments, at least 1, 2, 3, 4, 5, 6, 7, 8, 9, 10 or more administrations of the CK8 inhibitor occurs between consecutive administrations of the second therapeutic agent. In some embodiments, at least 1, 2, 3, 4, 5, 6, 7, 8, 9, 10 or more administrations of the second therapeutic of occurs between consecutive administrations of the CK8 inhibitor.

Additional therapeutic agents include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumor agents. All of these drugs affect cell division or DNA synthesis and function in some way. Additional therapeutics include monoclonal antibodies and the new tyrosine kinase inhibitors e.g. imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarbamide, idarubicin, ifosfamide, irinotecan, leucovorin, liposomal doxorubicin, liposomal daunorubicin, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, taxol and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to fludarabinetaurosporine, cycloheximide, actinomycin D, lactosylceramide, 15d-PGJ(2), and combinations thereof.

In a preferred embodiment, a CK8 inhibitor is administered in combination with doxorubicin.

### D. Methods of Selecting Subjects to Treat

In some embodiments, the subjects in need thereof are subject that have been selected based on a diagnosis and/or biomarker analysis. For example, in some embodiments, the subject in need thereof is selected for treatment based on having one or more of the cancers discussed above. Additionally or alternative, the subject can be selected for treatment because cells of the subject over- or aberrantly express CK8.

Methods of determining if cell of a subject has cells that over- or aberrantly expresses CK8 are known in the art. Generally, the methods include determining the localization, expression levels, or a combination thereof of CK8 mRNA, protein, or a combination thereof in a test sample of target cells or tissue isolated for a subject in question. Such methods may include, but are not limited to, microarray analysis, quantitative real-time RT-PCR, Northern blot, in situ hybridization, etc., for analysis of nucleic acid expression; and immunoaffinity based assays such as ELISAs, Western blots, immunohistochemistry, flow cytometry, and radioimmunoassays, and mass spectrometry based methods (e.g., matrix-assisted laser desorption ionization (MALDI), MALDI-Time-of-Flight (TOF), Tandem MS (MS/MS), electrospray ionization (ESI), Surface Enhanced Laser Desorption Ionization (SELDI)-TOF MS, liquid chromatography (LC)-MS/MS), etc., for analysis of protein expression.

Expression levels of CK8 in the test sample can be compared to a control. Exemplary controls include, but are not limited to, negative controls such as a sample of healthy tissue for the subject, preferably of the same cell type, adjacent to the target cells; and standards obtained from healthy subjects, such as subjects without cancer; and positive controls such as standards obtained from subjects that have been diagnosed with increased or aberrant expression of CK8. A control can be a single or more preferably pooled or averaged values of like individuals using the same assay. Subjects selected for treatment will typically have higher levels of CK8 than that of a negative control. The subjects selected for treatment can have levels of CK8 similar to that of a positive control. For example, in some embodiments the level of CK8 in a test sample is 25, 50, 75, 100, 125, 150, 175, 200, 250, 500, 750, 1,000 or more percent greater than the level of CK8 in a negative control.

An exemplary method includes (a) determining the level of CK8 in a test sample obtained from the subject; (b) comparing the level of CK8 in the test sample to the level of CK8 in a control; and (c) selecting the subject for treatment when the level of CK8 in the test sample is higher than the level of CK8 in the control. A method for selecting a subject for treatment can also include determining the levels of CK8 in a first test sample and a second test sample taken after the first sample, and selecting the subject for treatment when the level of CK8 in the second test sample is higher than the level of CK8 in the first sample.

### EXAMPLES

### Example 1

### CK8 Expression is Increased in ATC Cells

### Materials and Methods

Highly characterized thyroid cancer cell lines were obtained. Western blot and immunohistochemistry were used to determine Cytokeratin-8 (CK8) expression.

### Results

Cytokeratin-8 (CK8) is a type II intermediate filament, well studied for its role as a cytoskeletal (structural) protein. It is known to be highly overexpressed in a variety of malignant cells, and is used clinically and in research as an immunohistochemical and serum marker of malignancy (Shvero, et al., Oncol. Rep., 10(6):2075-8 (2003), Appetechia, et al., J. Exp. Clin. Cancer Res., 20(2):253-6 (2001)). In particular, elevated expression of keratin 8 (CK8) has been identified in several anaplastic thyroid cancer (ATC) cell lines, most notably the highly aggressive lines with fast population doubling time (Td).

Quantitative immunofluorescence was conducted to determine the expression level of CK8 in various cancer and normal cells. The results are illustrated in FIG. 1 (08-9515 a12 (tall cell), 12-2918 b1 (ATC), 08-8022 D4 (PTC+Adj Normal)).

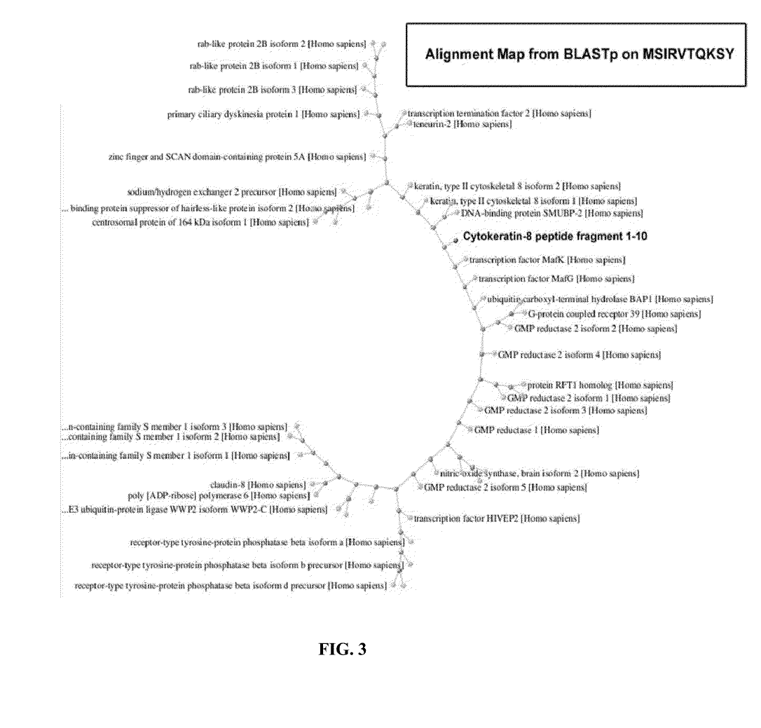

Fast growing thyroid cancer cell lines ATC1, 29T, and 11T had elevated expression of CK8. Slow growing cell lines 16T, FTC133, and 11T had undetectable CK8 levels. Population doubling time is illustrated in FIG. 2.

Additional studies showed that CK8 is overexpressed in human patient samples of papillary thyroid carcinoma, not detectable in matched normal thyroid and stroma, and highly overexpressed (at least 10 fold) in anaplastic thyroid carcinoma.

### Example 2

### Inhibition of CK8 Expression Reduces ATC Cell Proliferation

### Materials and Methods

Using a stable lentiviral-CK8 shRNA construct, a knockdown of CK8 was carried out in ATC1 cells. Following puromycin selection, culture wells were imaged daily to determine effect.

### Results

In ATC1 cells, scrambled lentivirus controls demonstrated expected recovery and proliferation following infection. Accordingly, no effect was observed with scrambled shRNA and GFP-only controls. Wells with no virus (negative control) underwent apoptosis. The CK8 lentivirus wells showed near total growth arrest of ATC cells. Escape was observed only after propagation of the senescent ATC1-CK8(low) cells for 8 weeks. At this point growth was restored and CKS western blot was nearly equivalent to wild type ATC1. These results indicate that more than simply being a biomarker for epithelial cancer, CK8 may play a direct role in anaplastic thyroid cancer progression.

### Example 3

### CK8 that has Bound an ATP Analogue, is Present in Highly Aggressive Anaplastic Cancer Cell Lines

### Materials and Methods

Activity-based protein profiling was carried out according to a method adapted from Bachovchin and Cravatt, Nat Rev Drug Discov.; 11(1):52-68 (2011), which is specifically incorporated herein in its entirety.

### Results

The 3D structure of CK8 is not well defined, and although it has been previously used as a marker for immunohistological studies, its function is poorly understood. CK8 can be expressed on the cell surface where it can be a plasminogen receptor, and is believed to form covalent bonds with membrane lipids, and interact with WIC molecules which may increase the metastasis of carcinoma cells. CK8 is also a cytoplasmic substrate for c-Jun N-terminal Kinase. Functional studies show that loss of p53 or RB function yields increased CK8 & androgen receptor (Mouse prostate CA model); CK8=MDR phenotype (human breast CA) cell line; Cyclin D1 overexpression=CK8/18 high; loss of CK8-P yields increased tumor progression (OSCC); CK8-P may drive hepatocellular CA; CK8 may have a signaling role in cell adhesion (Breast CA).

Experiments were designed to investigate the role of CK8 in thyroid cancer. First, the domain structure of CK8 was investigated. Using protein structure prediction software (Genesilico), PFAM0038 (Intermediate Filaments), EC 2.7.11.1 (non-specific serine/threonine protein kinase) p=2e-17 domains were identified in CK8. Other well-known members of this family include GSK3-b, Aurora Kinase A, Tau Tubulin Kinase 1 (TTK1).

Furthermore, CK8 knockout mice have decreased hepatocyte ecto-ATP activity (Satoh, et al., Med. Electron Microsc., 32:209-212 (1999). ATP-binding site prediction software (ATPint) was used to identify putative ATP binding sites. The results are presented in Table 1 below.

TABLE 1
CK8 Putative ATP Binding Sites

| Pos | Residue | Score | Prediction |
|---|---|---|---|
| 1 | M | 0.73865101 | INTERACTING |
| 2 | S | −0.20717273 | INTERACTING |
| 3 | I | −0.2897477 | INTERACTING |
| 4 | R | 0.48828637 | INTERACTING |
| 5 | V | −0.36039055 | INTERACTING |
| 6 | T | 0.2248876 | INTERACTING |
| 7 | Q | −0.35485945 | INTERACTING |
| 8 | K | −0.032110102 | INTERACTING |
| 9 | S | −0.49841468 | INTERACTING |
| 10 | Y | 0.7383555 | INTERACTING |

A homology tree based on an alignment map from BLASTp using MSIRVTQKSY (SEQ ID NO:X) as the query is presented in FIG. 3.

Phosphoproteomic profiling of a panel of ATC cell lines, dichotomized as fastgrowing (Td<34 hr, recapitulate aggressive cancer phenotype) versus slow-growing (Td>34 hr, recapitulate more indolent phenotype) was also carried out. Profiling included phospho-serine/threonine enrichment, phosphotyrosine enrichment, and activity based protein profiling using an ATP-analogue that biotinylates lysine residues near the binding site.

Using activity-based protein profiling (ABPP), it was demonstrated that active CK8 that has bound an ATP analogue, is present in 100% of highly aggressive anaplastic cancer cell lines (e.g. population doubling time <34 hrs), whereas there is no detectable active-CK8 in slow-growing anaplastic cancer cell lines. These data are confirmed by western blot and immunohistochemistry in multiple cell lines including highly aggressive cervical cancer (HeLa), thyroid (ATC1) and breast (MCF7) lines.

Sequence homology comparison using protein-protein BLAST demonstrates the c-terminal putative ATP-binding region (CK8 aa 120-240) shares significant homology (95% identity match) with tau tubulin kinase-1 (TTK1) aa375-490, and CK8 aa 25-260 has 89% homology with TTK1 aa 251-484.

Interestingly, CK8 also shares stretches of sequence with several other known cancer/cell cycle actors including secreted frizzled-related protein 3 precursor (75% homology), claudin-8 (67%), transcription factor HIVEP2 (100%), DNA-binding protein SMUBP2 (100%), and Teneurin-3 (88%).

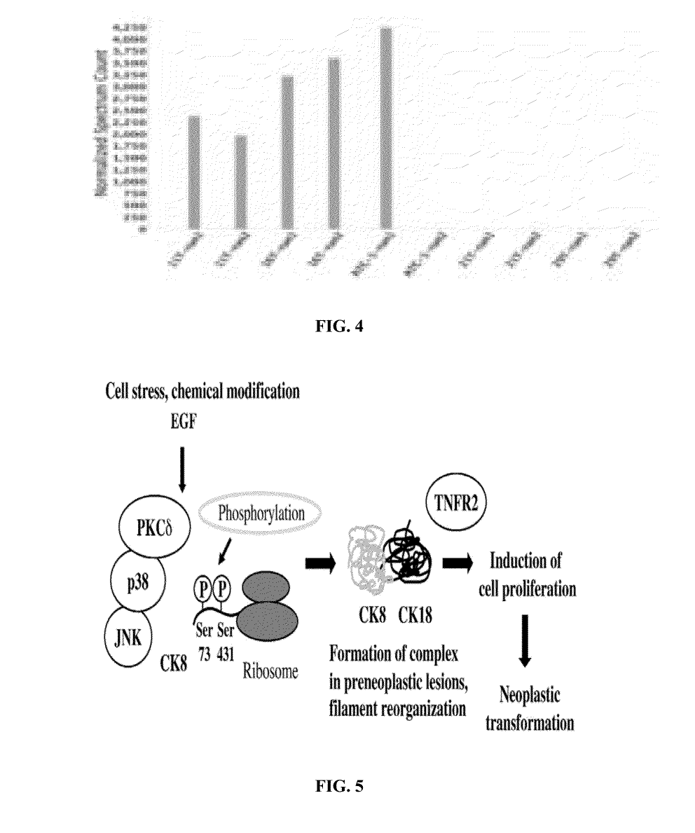

Phosphotyrosine (pY) Immunoprecipitation (IP)-LC/MS-MS was used to investigate the differential phosphorylation events in fast-growing and slow-growing thyroid cancer cells lines. Tubulin-beta was sequenced in all replicates (38% coverage, 12 unique peptides). New phosphorylation events were found in 5/6 fast-growing lines, versus 0/4 slow-growing lines (p=0.0010). The results are illustrated in FIG. 4.

Collectively, these studies indicate a role for cytokeratin-8 as an active consumer of ATP and an active tumor promoter in anaplastic thyroid carcinoma, and likely many other highly aggressive cancer types. Preliminary evidence indicates a mechanism of action for CKS, illustrated in FIG. 5, that involves the SRY2/P27/alternative EGFR pathway. The model is compatible with others investigations that failed to show efficacy for EGFR kinase blockade, despite EGFR overexpression in anaplastic thyroid cancer.

Unless defined otherwise, all technical and scientific terms used herein have the same meanings as commonly understood by one of skill in the art to which the disclosed invention belongs. Publications cited herein and the materials for which they are cited are specifically incorporated by reference.

### Example 4

### Genomic Analysis

### Materials and Methods

Fifteen solid tumor types from TCGA with RNAseq gene expression data available were analyzed for KRT8 expression patterns.

Human Patient Cohort Selection.

Informed consent was waived by the IRB for this study under 45 CFR §164.512(REF). Department of Otolaryngology and Pathology records were reviewed to identify all consecutive patients undergoing either biopsy or tumor resection with a histologic diagnosis of ATC from 2003-2013 at Georgia Regents University (previously “Georgia Health Sciences University,” previously “Medical College of Georgia”). A convenience sampling of patients from the same time frame, diagnosed with classic histology PTC were also included. Exclusion criteria were lack of available FFPE archival specimen. Survival and recurrence data were determined from a combination of review of medical records, personal communication, social security death database, and public records. Patients with recurrent cancer or previous malignancy were included, but were excluded from survival and outcomes analyses. Patient medical charts were also reviewed for relevant demographic and pathologic information, staging, and treatment.

General Laboratory Reagents and Chemicals.

All reagents and chemicals used were purchased from Fisher Scientific (Hampton, N.H.) unless otherwise specified below.

Genomic Data Analysis.

The Cancer Genome Atlas (TCGA) RSEM-normalized RNA sequencing data for all primary tumor and tissue normal samples from 15 solid-tumor cancers (Bladder Urothelial Carcinoma-BLCA, Breast invasive carcinoma-BRCA, Cervical squamous cell carcinoma and endocervical adenocarcinoma-CESC, Colon adenocarcinoma-COAD, Glioblastoma multiforme-GBM, Head and Neck squamous cell carcinoma-HNSC, Kidney renal papillary cell carcinoma-KIRP, Liver hepatocellular carcinoma-LIHC, Lung adenocarcinoma-LUAD, Lung squamous cell carcinoma-LUSC, Pancreatic adenocarcinoma-PAAD, Sarcoma-SARC, Skin Cutaneous Melanoma-SKCM, Papillary Thyroid carcinoma-THCA, Uterine Corpus Endometrial Carcinoma-UCEC) were downloaded using TCGA-Assembler. Normalized gene expression data was log transformed prior to differential gene expression analysis. In order to understand KRT8 expression profile in solid tumors, we plotted the log fold change between cancer and normal for each tumor type (FIG. 1a). LIMMA package in a custom R script was used to test for differential gene expression between cancer and normal samples for each of the 15 cancer types. One limitation of fold-change based analyses is that heterogeneous sub-populations can be obscured. Therefore, distribution of normalized gene expression values was visualized on a per-case basis for cancer and normal samples for each tumor type (FIG. 7). Additional tools for visualizing and summarizing TCGA genomic data were also accessed through the MSKCC cBioPortal.org web-based interface. Available datasets were evaluated on cBioPortal.org that met the following inclusion criteria: solid tumor cancer types, n>100 cases, annotated with copy number and mutation data. In cases where more than one dataset from a given cancer type was available the largest dataset was used. Datasets included were: adrenocortical carcinoma, bladder urothelial carcinoma, brain lower grade glioma, breast invasive carcinoma, cervical-squamous cell carcinoma and adenocarcinoma, colorectal adenocarcinoma, cutaneous melanoma, glioblastoma multiforme, head and neck squamous cell carcinoma, hepatocellular carcinoma, lung adenocarcinoma, lung squamous cell carcinoma, pancreatic adenocarcinoma, pheochromocytoma and paraganglioma, prostate adenocarcinoma, renal clear cell carcinoma, renal papillary cell carcinoma, stomach adenocarcinoma, thyroid carcinoma, and uterine endometrial carcinoma. Genomic alterations in KRT8 (mutation, deletion, amplification) were summarized by cancer type and visualized using the cBioPortal.org interface.

Statistical Analysis.

Flow cytometry data were converted to FCS format and visualized in FlowJo v9 (FlowJo LLC, Ashland, Oreg.). Genomic data were analyzed using LIMMA (Ritchie et al. 2015) package in a custom R script. All other statistical analyses were performed using SPSS version 23.0 (IBM Corp.) and were two-tailed where appropriate. A p value of 0.05 was set for determining significance. Graphs for data visualization were produced using SPSS (IBM Corp., Carey, N.C.), MS Excel 14.4 (Microsoft, Redmond, Wash.), LIMMA, and FlowJo (FlowJo LLC).

### Results

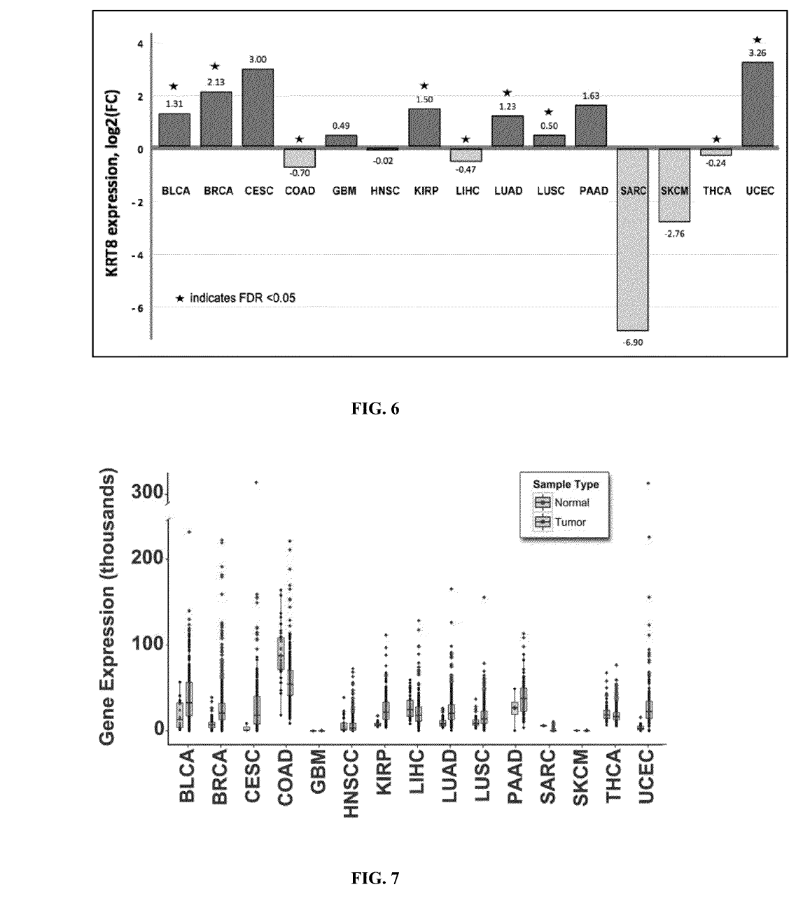

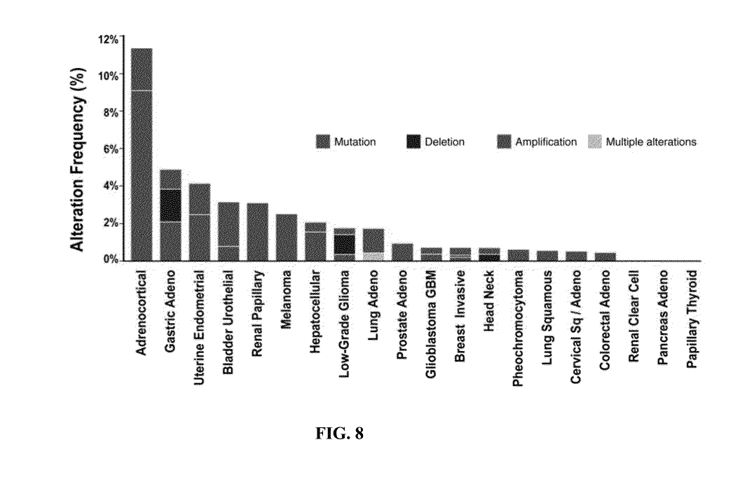

The mean KRT8 expression for matched tumor:normal pairs was compared within each tumor type. Six tumor types had significantly elevated mean KRT8 expression ratio (FDR<0.05): Bladder, Breast, KIRP, LUAD, LUSC, UCEC, while three had significantly decreased KRT8 expression in tumor versus normal, summarized in FIG. 6. As mean gene expression comparisons can miss a population with heterogeneous distribution of expression, KRT8 expression for each cancer case was next plotted (FIG. 7). These data visualizations clearly show that within most cancer types there is a substantial population subset with elevated KRT8 expression. Only GBM, sarcoma, SKCM, and papillary thyroid cancer showed no KRT8-elevated subpopulations. It should be noted that the TCGA thyroid cancer dataset did not include any ATC patients. Next, genomic datasets including 5,625 patients across 20 solid cancer types were analyzed and visualized using MSKCC's web-based interface available at cBioPortal.org. Genomic alterations of KRT8 were present in 0 to 4.9% (mean 1.5%) of cases. The three solid cancers with the highest prevalence of KRT8 genomic alterations were adrenocortical carcinoma, stomach adenocarcinoma (4.9%), and uterine endometrial carcinoma (4.1%). Contrarily, no KRT8 genomic alterations were detected among pancreatic adenocarcinoma, renal clear cell carcinoma, or papillary thyroid carcinoma (all 0%). Among the 73 cases with KRT8 genomic alterations of any type, mutation (almost all missense) was the most common alteration, present in 34/73 (47%) cases followed by amplification (27/73, 37%). Genomic deletion of the keratin-8 locus was relatively infrequent (11/73, 15%).

### Example 5

### Patient Demographics and Keratin-8 Expression

### Materials and Methods

Immunohistochemistry.

TMA and control slides were deparaffinized with xylene followed by graded ethanol washes and rehydrated in TBS. Antigen retrieval was performed using Diva Decloaker (Biocare Medical, Concord, Calif.) for 1 minute in a 100° C. pressure cooker followed by endogenous peroxidase block using 0.03% hydrogen peroxide (Peroxidase Block) (Envision System, Dako, Carpenteria, Calif.) for 30 minutes. Non-specific antibody binding was then blocked with 0.3% bovine serum albumin for 30 minutes at room temperature. Following these steps, slides were incubated with primary antibody at 4° C. overnight: Keratin-8 (mouse monoclonal, clone 4.1.18; Millipore); cleaved caspase-3 (cCas3, mouse monoclonal). Subsequently, slides were incubated with goat anti-mouse secondary antibody conjugated to a horseradish peroxidase-decorated dextran polymer backbone (Envision; DAKO North America, Carpenteria, Calif.) for one hour at room temperature. Bound antibody was subsequently visualized using diaminobenzidine (DAB) chromagen followed by acidified hematoxylin counterstain.

Cell Line Processing/Embedding and Tissue Microarray Construction.

Cell lines for paraffin embedding were grown to 80% confluence in T175 tissue culture flasks (Corning Life Sciences.), and fixed in situ with 10% formalin and released using a Falcon 18 cm cell scraper (Fisher Scientific). Cells were then pelleted at 500 g, the supernatant aspirated and cells suspended in lukewarm 1% agarose and allowed to gel. The agarose cell pellet was then embedded in standard histologic paraffin (Fisher Scientific) and the cell block used in the subsequent tissue microarray (TMA). The TMA was assembled from cell-line blocks, clinical formaldehyde fixed paraffin embedded (FFPE) surgical samples including ATC patient and normal control tissues. H&E counterstained slides from all blocks were reviewed at 10× and 20× by light microscopy, and marked with permanent fine-tip marker to select representative areas of tumor to be cored. The cores were placed into the recipient microarray paraffin block using an ATA100 Tissue Microarrayer (Veridiam, Ocean Side, Calif.). All tumors were represented with two-fold redundancy using 0.9 mm cores. Following assembly and annealing of cores, 7 μm sections were cut on a microtome, mounted to silanized slides, and used for IHC. Every 15^{th} section was H&E stained to confirm tumor spot retention and histology confirmation. Cells for Cas-9 immunohistochemistry, cytoskeleton morphology, and TUNEL staining were prepared using 8-well chamber slides (Millipore), fixed in 10% formalin for 5 minutes, rinsed with PBS and processed.

Quantitative In Situ Protein Expression Measurement.

Protein expression was determined using a multispectral imaging workstation (Nuance FX, Perkin Elmer, Akron, Ohio) attached to a Zeiss Axiophot I microscope (Carl Zeiss Microscopy, Thornwood, N.Y.). Images were acquired at 40× magnification with 1×1 binning, gain set to 1.0 and cube auto-expose used to control image capture time. Pure dye spectral libraries were created for DAB chromagen and hematoxylin counterstain (Supplemental Data). Images were spectrally unmixed to individual dye channels based on the spectral libraries, allowing quantitative measurement of protein expression independent of hematoxylin counterstain intensity. Nuance values were recorded as average optical density (OD), which averages DAB (signal-specific) intensity across multiple pixels weighted by area. We have previously derived¹⁸ that nuance values correlate to target concentration in a log-log relationship represented by the linear equation: log₁₀(NuanceScore)=M×log₁₀(TargetConcentration)+B, where B is the Y intercept and M is slope. Therefore, a relative expression scale (1-1000) was created using cell line and tissue controls, with 1 arbitrarily defined as the lowest nuance score obtained, and 1000 set as the highest score. These scale scores and the nuance scores were log transformed, an equation fit to this line, and used to transform all nuance average OD values onto the 1-1000 scale.

### Results

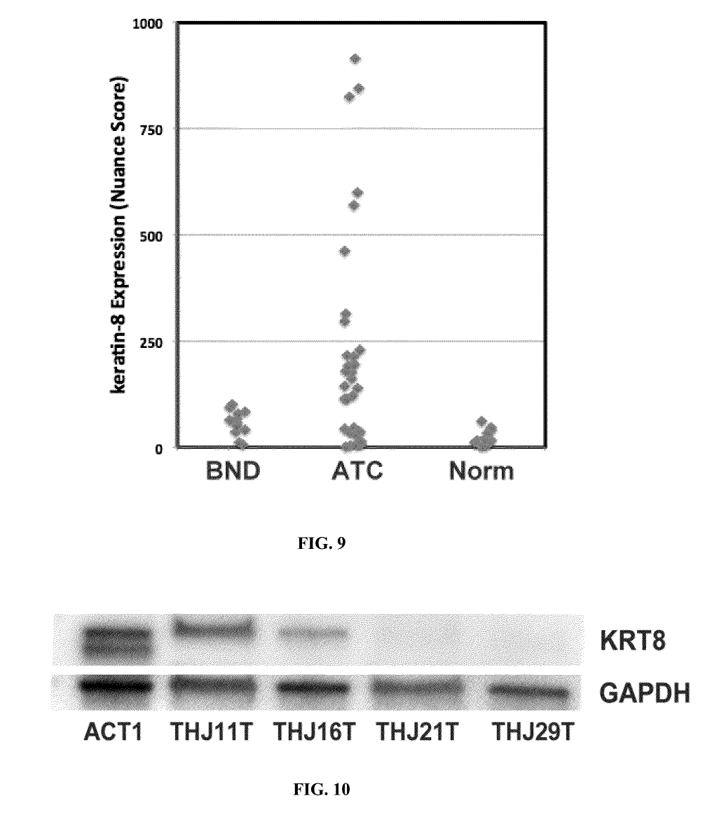

Keratin-8 expression was evaluated in patients with ATC. There were 19 patients included for quantitative IHC analysis of keratin-8 expression level, consisting of nine patients with ATC, six patients with classical papillary thyroid cancer (PTC), and control tissue from four patients with benign multinodular goiter (BNG). Patient-matched adjacent histologically normal thyroid tissue was available in 7 patients. Tissue samples were assembled into a tissue microarray, and subjected to immunohistochemistry using a mouse monoclonal anti-keratin-8 antibody. Keratin-8 expression was determined quantitatively by multispectral imaging (Nuance, Perkin-Elmer) on a 1-1000 scale. Keratin-8 expression was not confined to a particular type of sample, and was primarily cytoplasmic in distribution for most samples. Overall there was increased keratin-8 expression in ATC, compared to both PTC, and normal thyroid. Keratin-8 expression in clinical samples is summarized in FIG. 9.

### Example 6

### Keratin-8 Expression In Vitro

### Materials and Methods

Patient-Derived ATC Cell Lines.

Five well-characterized anaplastic thyroid cancer cell lines were obtained as gifts from Dr. J. Copland (Mayo Clinic, Jacksonville Fla.; THJ11T, THJ16T, THJ21T, and THJ29T cell lines) and Dr. S. Ohata (Tokushima University, Japan; ACT1 cell line). Of note, many supposedly “anaplastic thyroid” cell lines previously described in the literature have now been demonstrated to be contaminants and not of thyroid origin, with only a few exceptions including the Mayo cell lines. These lines (THJ11T, THJ16T, THJ21T, and THJ29T) were derived at Mayo Clinic—Jacksonville (FL) from human ATC primary tumors, and have been previously characterized. Additional epithelial cancer cell lines known to display rapid in vitro growth (HeLa and MCF7) were obtained from the American Tissue Culture Collection (ATCC). All cell line identities used for these experiments were confirmed in our laboratory by independent STR analysis after initial propagation and storage of parent stocks in liquid nitrogen, and STR analysis is repeated annually for cell lines in use (see Supplemental Data). Cell lines were propagated at 5% CO₂ in a humidified tissue culture cabinet at 37° C. in standard RPMI media supplemented with 10% fetal bovine serum (FBS), 100 U/ml Penicillin, 100 μg/ml Streptomycin and 0.25 μg/ml Amphotericin B. Additionally, media used for lines THJ11T, THJ16T, THJ21T, and THJ29T was supplemented with 1× non-essential amino acids, 1 mM Sodium Pyruvate, and 10 mM Hepes.

Western Blot.

Adherent cells at 85% confluence were rinsed in PBS, then lysed with modified RIPA buffer consisting of 50 mM Tris-HCl [pH 7.4], 150 mM NaCl, 5 mM EDTA, 1% NP-40, 0.1% sodium dodecyl sulfate (SDS), 0.5% sodium deoxycholate, PhosStop phosphatase inhibitor cocktail (Roche Diagnostics, Indianapolis, Ind.), and complete proteinase inhibitor cocktail (Roche Diagnostics). Lysate total protein concentration was determined by Fourier transform infrared spectrophotometer (Direct Detect FTIR, EMD Millipore, Bedford, Mass.). 5 μg of total protein was added to equal volume laemelli buffer containing beta-mercaptoethanol (BioRad Laboratories, Hercules, Calif.) and heat treated at 100° C. for 4 minutes. Proteins were resolved by electrophoresis in AnyKD Mini-Protean TGX SDS-polyacrylamide gels, transferred polyvinylidene difluoride (PVDF) membrane in Tris-glycine buffer, and blocked in 3% BSA-TBST buffer (3% bovine serum albumin (BSA), 25 mM Tris-HCl [pH 8.0], 125 mM NaCl, and 0.5% Tween 20) for 1 h at room temperature. The membranes were probed with primary antibodies then washed in TBST and incubated with goat-anti rabbit or goat-anti mouse antibody conjugated to a horseradish peroxidase-decorated dextran polymer backbone (Envision System, Dako, Carpenteria, Calif.) overnight at 4° C. Membranes were washed in TBST and then incubated with SuperSignal West chemiluminescent substrate (Thermo Scientific) and imaged.

### Results

Keratin-8 expression was determined in five well characterized patient-derived ATC cell lines (ACT1, THJ11T, THJ16T, THJ21T and THJ29T) by immunohistochemistry and western blot, using two different monoclonal mouse antibodies with specific reactivity to keratin-8 (FIG. 10). There was concordance between immunohistochemistry and western blot for each line. Similar to the human clinical cohort, there was a heterogeneous expression of keratin-8 among ATC cell lines, with 3/5 (60%) having strong expression and 2/5 (40%) having almost non-detectable keratin-8 expression.

### Example 7

### RNA-Interference Mediated Knockdown of Keratin-8

### Materials and Methods

shRNA Lentivirus Knockdown.

Commercially available replication-incompetent lentivirus shRNA constructs targeting the KRT8 gene, which encodes the cytokeratin-8 protein, and a puromycin resistance gene were purchased (sc-35156-V, SantaCruz Biotechnology, La Jolla, Calif.) and used per manufacturer's instructions. The shRNA sequences for this lentivirus are proprietary, but reported as a pooled mixture of three separate shRNAs each targeting KRT8. Briefly, cells were plated equally in 6-well or 12-well plates at 3×10⁵ and 1×10⁵ cells/well, and allowed to reach 50% confluence (approximately 24 hours). The media was then exchanged for transduction media containing 10% FBS, polybrene (previously experimentally titrated to 2 μg/ml) and lentiparticles at an MOI of 1.0, and incubated for 72 hours at 37° C. The transduction media was then exchanged for media with 10% FBS and puromycin 4 μg/ml, and changed every 72 hours. Negative controls consisted of identical lentiparticles but encoding a nonsense shRNA sequence (SantaCruz Biotechnology). Lentiparticle transduction efficiency was experimentally determined using control lentiparticles encoding green fluorescent protein (GFP) with puromycin resistance gene. Knockdown efficiency was determined by rtPCR for keratin-8 mRNA.

Flow-Cytometry Based Cell Viability and Apoptosis Assays.

Cells were seeded in 12-well tissue culture plates (Corning Life Sciences) at 30% confluence. Twenty-four hours later (at 50% confluence) lentiviral transduction (SantaCruz sc-35156-V) was performed as described above at MOI=1. Following media exchange to selection media, cells were allowed to grow for 96 hours, the media aspirated and analyzed separately, and the adherent cells released with trypsin. Cells were gently centrifuged to pellet and resuspended in 1.0 ml of RPMI+10% FBS, and a 100 μl aliquot used to perform Annexin V/Apoptosis/Viability detection per manufacturer's instructions (MUSE Annexin V/Apoptosis Assay, EMD Millipore, Billerica, Mass.). Aspirated supernatant (containing detached cells) was pooled to combine 3-4 experimental replicates, centrifuged to pellet cells and debris, resuspended and analyzed as above. Experiments were performed with at least three biological replicates, and two technical replicates of each sample. The MUSE instrument was set at a constant sample flow rate of 0.18 μl/sec, with a target of 4000 events, therefore the [time to acquisition] data field was used to calculate original sample concentration (cells/μl).

### Results

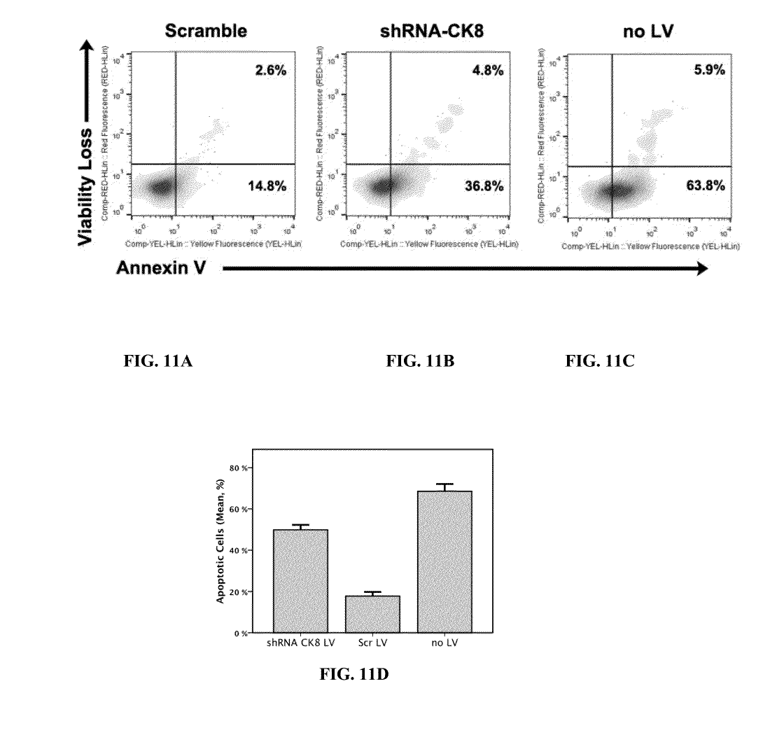

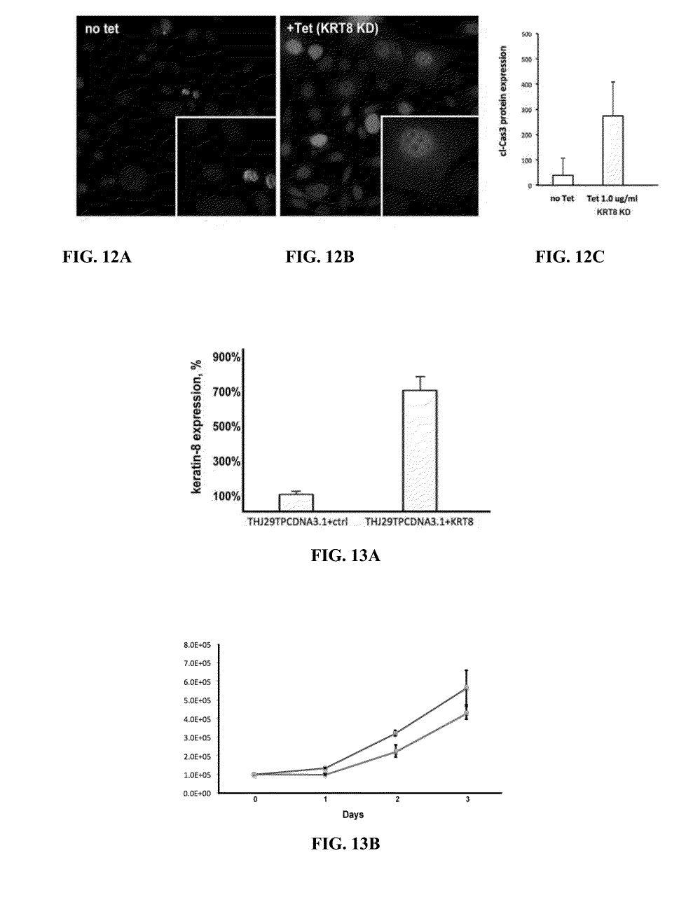

Replication incompetent lentivirus was used to stably express shRNA specific to KRT8 gene transcript in ACT1 (keratin-8 high expressing) cells, along with a puromycin resistance marker, resulting in 90% reduction in KRT8 mRNA expression (FIGS. 11A-11C). Following transduction, cells were maintained for 96 hours in media with 4 μg/ml puromycin and then analyzed for cell viability and apoptosis using a flow cytometry-based apoptosis assay (EMD Millipore Cell Viability and Apoptosis Kit). Controls included ACT1 cells transduced with nonsense scramble shRNA encoding lentivirus (with puromycin resistance gene), and wild type ACT1 cells without lentivirus (and thus lacking puromycin resistance). Cells subjected to RNAi mediated keratin-8 knockdown had a decrease in number of viable, non-apoptotic cells recovered (mean 253.2×10X±SD 10.6 cells/μl) compared to both scramble controls (1038.6×10X±53.7 cells/μl) and untreated controls lacking puromycin resistance (300.4×10X±13.6 cells/μl), p<0.001 for all comparisons (FIG. 11D). This loss of viability was partially mediated by increased apoptosis, with 49.9%±SD 2.4 of keratin-8 knockdown cells in early or late apoptosis compared to 17.8%±2.0 of scramble-shRNA control cells, p<0.001). Flow cytometry results are summarized in FIGS. 11A-11C.

### Example 8

### Tet-Inducible Keratin-8 Knockdown and Apoptosis

### Materials and Methods

Tetracycline-inducible shRNA knockdown. Because of the severe decrease in cell viability following keratin-8 knockdown, stable clones were not able to be selected and propagated for further study. Therefore, an inducible keratin-8 knockdown ATC line (designated ACT1^{+tetR+ck8shRNA#c}) was created by co-transduction of ATC cell lines with lentiparticles containing the TetR regulatory element and neomycin resistance gene (GenTarget, San Diego Calif.), and a custom tetracycline-inducible shRNA lentiviral construct. Six shRNA sequences were designed to target keratin-8 transcript variant 1, GenBank NM_001256282.1. Lentivirus containing these shRNAs under suCMV promoter control with two upstream tet-responsive repressor elements, and a puromycin resistance/GFP fusion cassette under RSV promoter (AMSBIO, Cambridge Mass.) were assembled. This design allows use as either an ordinary shRNA construct (in the absence of TETR protein), or as a tet-on system (when co-transduced with LV particles encoding the TETR protein). Replication incompetent lentiparticles were prepared by co-transfecting plasmids containing the shRNA-resistance-GFP construct and a packaging plasmid into HEK293 cells according to manufacturer recommendations. Cell media containing the lentiparticles was then purified by ultracentrifugation and frozen at −80° C. until use. Briefly, cells were grown to 50% confluence in 6-well tissue culture plates (Corning Life Sciences, Tewksbury, Mass.). Lentivirus was diluted into RPMI with 10% FBS and 2 μg/ml polybrene, applied at a multiplicity of infection=1.0 and incubated at 37° C. for 48 hours. Subsequently the transduction media was aspirated and replaced with RPMI supplemented with 10% FBS and puromycin at 4 μg/ml (Fisher Scientific). Knockdown efficiency was determined by rtPCR for keratin-8 mRNA, and the best shRNA sequence (shRNA#c) was selected for further experiments. Stable double-transduction clones were then selected in DMEM+10% certified tetracycline-free FBS, neomycin and puromycin, and confirmed by rtPCR and western blot (supplemental data). Transduction efficiency was determined using GFP expression under fluorescent microscopy. shRNA expression and keratin-8 knockdown was then subsequently induced by addition of 1 ug/ml tetracycline to cell culture media. Knockdown efficiency with and without tetracycline was determined by rtPCR.

Cell Morphometry/Cytoskeletal Visualization and TUNEL Staining.

A fluorescent apoptosis TUNEL assay (Abcam) was used to quantify fragmented DNA, with the standard protocol modified to allow cytoskeletal visualization: ACT1^{+tetR+ck8shRNA#c} cells were grown in 4-well chamber slides (Corning) in RPMI media supplemented with certified tetracycline-free 10% fetal bovine serum (FBS), 100 U/ml Penicillin G, 100 μg/ml Streptomycin and 0.25 μg/ml Amphotericin B. Cells were brought to 50% confluence, at which point the media was exchanged for identical media containing 1 ug/ml tetracycline. Media was subsequently exchanged every 48 hours without passaging of the cells. At time points (0, 24, 48, 72 hours) replicate samples were fixed with 10% formalin×15 minutes, permeabilized with proteinase-K, and incubated with TdT enzyme and Br-dUTP followed by Rhodamine-labelled anti-BrdU antibody. Slides were then incubated with phalloidin-alexa488 conjugate (emission peak 519 nM) to label the F-actin cytoskeleton, followed by DAPI to label nuclei. Imaging was performed using the Nuance MSI system at 40× and a triple-emission fluorescent filter set (Chroma) at 20 nM wavelength intervals from 460 to 720 nM. Similar to protein expression quantitation, pure-dye spectral libraries were created and used to spectrally unmix the resulting image cubes. For fluorescent microscopy, nuance scores were recorded as average scaled counts per second, which corrects for exposure time and reflects a weighted average across the designated area of interest. Nuance scores were log-log transformed as above to a 1-1000 scale. Additionally, for the TUNEL assay, the percent of tumor cell nuclei positive for Br-dUTP was calculated. For each condition 3-5 representative high powered fields were imaged and analyzed.

### Results

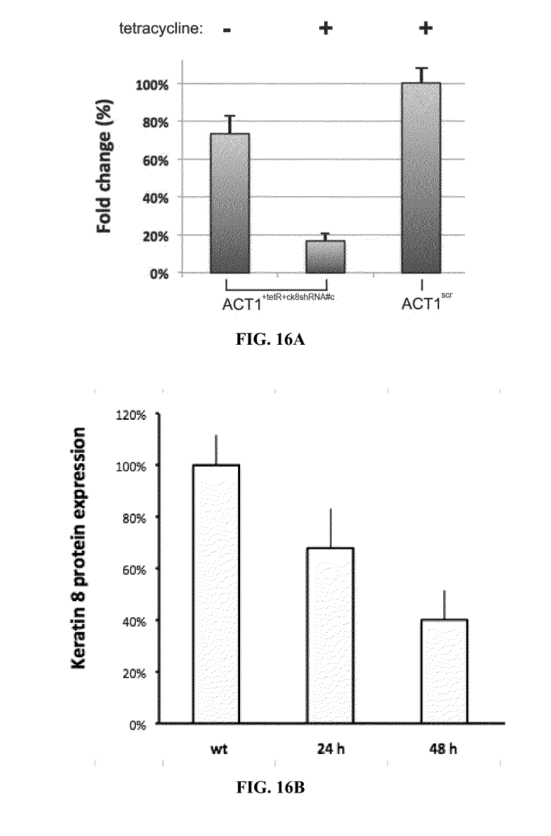

Given the substantial deleterious effect on cell survival following KRT8 knockdown, a tetracycline-inducible shRNA lentiviral construct was created. This model was used to further interrogate KRT8 knockdown effects in ACT1^{+tetR+ck8shRNA#c} cells by immunohistochemistry for cleaved caspase-3 (cCas3) (FIGS. 12A-12B and FIGS. 16A-16B), and TUNEL staining. Cells grown with tetracycline 1.0 ug/ml resulted in 80% reduction in KRT8 expression compared to scrambled control at 48. Cells were labeled with phalloidin to visualize the F-actin cytoskeleton, and fragmented DNA was visualized using fluorescent TUNEL staining. A phenotypic effect was evident in the KRT8 knockdown cells, beginning at 24 hours and becoming most prominent at 48 hours. This was characterized by a generalized increase in mitotic figures, along with nuclear coalescence of the actin cytoskeleton in a subset of cells. These cells also displayed condensation of the nucleus and loss of nucleolar definition.

The increase in apoptosis was accompanied by an elevation in cleaved caspase-3 (cCas3) at 48 h, (mean±SD) in knockdown ACT1 cells compared to no-tetracycline controls (±) (FIG. 12C).

### Example 9

### KRT8 Overexpression

### Materials and Methods

KRT8 Plasmid Transduction.

THJ29T cells were transduced with PCDNA3.1 plasmid containing the neomycin resistance gene for selection, plus either scrambled cDNA or the full-length KRT8 coding sequence. Following selection in media supplemented with neomycin, stable clones were selected, propagated, and mRNA and protein harvested to confirm KRT8 overexpression using rt-PCR and western blot for KRT8 as above. These cells (designated THJ29T^{PCDNA3.1+Scr} or THJ29T^{PCDNA3.1+KRT8}) were analyzed as above for apoptosis and cell proliferation both at standard conditions and under redox stress conditions by adding 100 uM hydrogen peroxide to standard media for 24 hours.

### Results

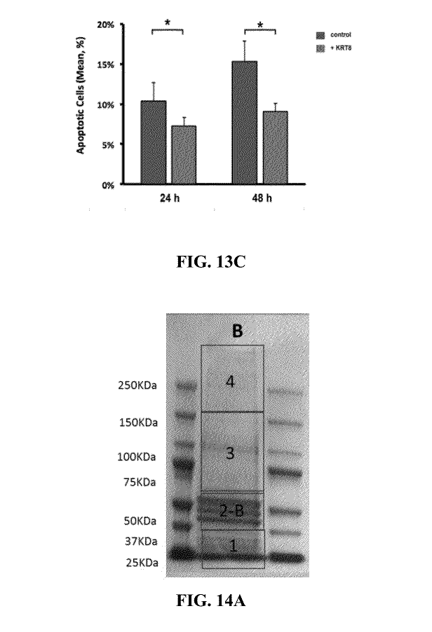

To investigate the effects of forced overexpression of KRT8, THJ29T (KRT8 low-expressing) cells were transduced with either scrambled plasmid or plasmid encoding the KRT8 transcript variant 3 mRNA under promoter control. Following selection in antibiotic media, clonal sub-cultures of cells were plated at controlled density in replicate 6-well plates and assayed at 24, 48, and 72 hours post-seeding. At 48 and 72 hour time points, cells were also assayed for apoptosis by Annexin-V flow cytometry (as above). There was no significant difference between scramble controls and KRT8 overexpressing cell populations (data not shown), however when experiments were repeated under redox stress conditions (adding 100 uM hydrogen peroxide to media), KRT8 overexpression was associated with significant reduction in apoptosis, with 15.3% (±SD 2.6) of THJ29T^{PCDNA3.1+Scr} cells being apoptotic versus 9.1% (±1.0) THJ29T^{PCDNA3.1+KRT8} cells at 72 h (FIGS. 13A-13C, p<0.001).

### Example 10

### Co-Immunoprecipitation/Annexin-A2 Binding

### Materials and Methods

Immunoprecipitation.

ACT1 cells were grown to 80% confluence, collected by cell scraper without trypsinization, and centrifuged at 500×g for 5 minutes to create a cell pellet. Cell pellets were subsequently stored at −80° C. and then processed as a group. Pellets were suspended in lysis buffer (25 mM Tris-HCl pH 7.4, 150 mM NaCl, 1 mM EDTA, 1% NP-40 and 5% glycerol) and homogenized using a BioPulverizer. Lysate was then centrifuged and the supernatant passed through a Zeba desalting spin column (89891, Thermo). Immunoprecipitation was performed by incubating the lysate with magnetic beads pre-conjugated to either keratin 8 or annexin A2 or control (non-immune rabbit IgG fraction, Abcam) followed by magnetic bead capture. Beads were rinsed three times, and eluted using either high-pH elution (for mass spec downstream) or 3× non-reducing lamelli buffer (for western blot).

Mass Spectrometry.

Following immunoprecipitation, IP product was run on a polyacrylamide gel (each replicate/sample as a separate lane) and coomassie blue stained to identify major protein bands. 4 bands from each sample were excised, and in-gel tryptic digestion performed in 2 M urea, 20 mM Tris, pH 8.0 and trypsin at 37° C. overnight with enzyme to protein ratio of 1:50. Peptides were eluted with 50% acetonitrile with 0.1% trifluoroacetic acid, Speedvac concentrated, and re-dissolved in aqueous 2% acetonitrile with 0.1% trifluoroacetic acid. A nanoflow liquid chromatograph (U3000, Dionex, Sunnyvale, Calif.) interfaced with an electrospray ion trap mass spectrometer (LTQ-Orbitrap, Thermo, San Jose, Calif.) was used for tandem mass spectrometry peptide sequencing experiments, as previously described¹⁹. Each sample was first loaded onto a trapping column (5 mm×300 μm ID packed with C18 reversed-phase resin, 5 μm, 100 Å) and washed for 8 minutes with aqueous 2% acetonitrile with 0.04% trifluoroacetic acid. The trapped peptides were then eluted onto the analytical column, (C18, 75 μm ID×15 cm, Pepmap 100, Thermo). The 120-minute gradient was programmed as: 95% solvent A (2% acetonitrile+0.1% formic acid) for 8 minutes, solvent B (90% acetonitrile+0.1% formic acid) from 5% to 50% in 90 minutes, increasing from 50% to 90% B in 7 minutes, then held at 90% for 5 minutes. Re-equilibration was achieved by decreasing solvent B from 90% to 5% in 1 minute and holding at 5% B for 10 minutes. The flow rate for the analytical column was 300 nl/min. Five tandem mass spectra were collected in a data-dependent manner following each survey scan. The MS scans were acquired in the Orbitrap with AGC target set to 1,000,000 to obtain accurate peptide mass measurements, and the MS/MS scans were acquired in the linear ion trap using with AGC target set to 30,000 and 60 second exclusion for previously sampled peptide peaks.

Protein Identification.

Tandem mass spectra were analyzed using Mascot (Matrix Science, London, UK; version 2.2.04) and Sequest (Thermo Fisher Scientific, San Jose, Calif., USA; version SRF v. 3) both designated to search the Uni-Prot human database (version prot_20120711) assuming the digestion enzyme trypsin¹⁹. Up to three missed tryptic cleavages were allowed. Sequest and Mascot were searched with a parent ion tolerance of 1.2 Da and fragment ion mass tolerance of 0.80 Da. Variable modifications included carbamidomethylation (Cys), oxidation (Met), and desthiobiotinylation (Lys). Scaffold (version 4.3.4, Proteome Software Inc., Portland, Oreg.) was used to catalogue MS/MS based peptide and protein identifications. Peptide identifications were accepted if they could be established at greater than 95.0% probability by the Peptide Prophet algorithm (Keller, A et al Anal. Chem. 2002; 74(20):5383-92). Protein identifications were accepted if they could be established at greater than 95.0% probability and contained at least 2 identified peptides. Protein probabilities were assigned by the Protein Prophet algorithm (Nesvizhskii, Al et al Anal. Chem. 2003; 75(17):4646-58). Proteins that contained similar peptides and could not be differentiated based on MS/MS analysis alone were grouped to satisfy the principles of parsimony. These parameters yielded a 0.3% false discovery rate (FDR) for peptide mapping and a 0.1% FDR for protein identification.

### Results

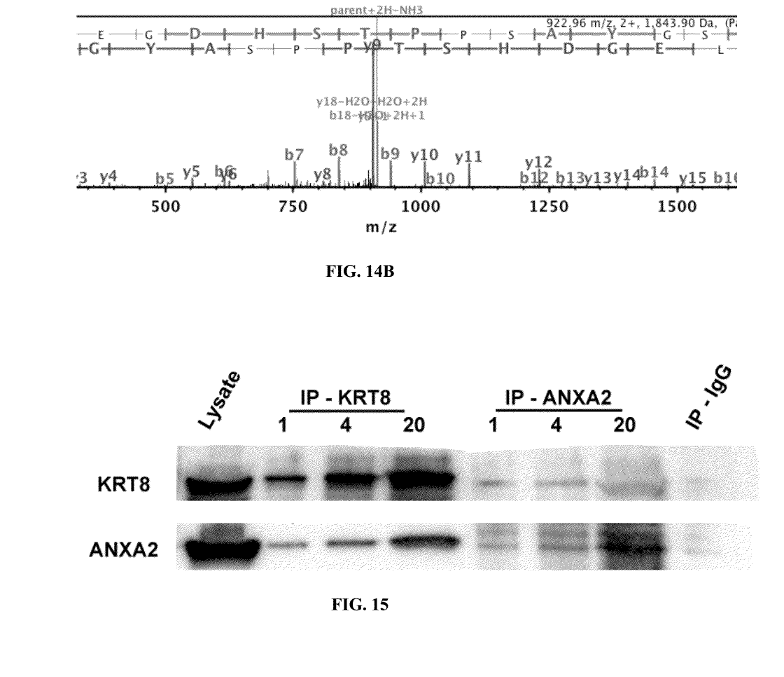

ACT1 whole-cell lysate was probed with anti-keratin-8 antibody covalently bonded to magnetic beads, rinsed multiple times, and eluted. The immunoprecipitation eluate was then subjected to PAGE/HPLC/tandem mass spectrometry to identify potential binding partners. As expected, several previously confirmed or suspected keratin-8 binding partners were reliably sequenced in multiple replicate samples including keratin-18, fibronectin, periplakin, sequestosome-1, and tight-junction protein ZO1. To our knowledge none of these would directly explain the observed interactions between keratin-8 and apoptosis resistance in anaplastic thyroid cancer cell lines (FIGS. 14A-14B). A novel potential binding partner, annexin-A2, was also sequenced with high degree of certainty in multiple replicate samples, spurring further investigation. Annexin A2 was reliably sequenced in 5/9 MS/MS runs, with a median 8 unique spectra (range 2-33) resulting in direct sequencing of mean 25.8% of the protein (range 6.8-54). Using the peptide prophet algorithm, the protein identification probability for annexin-A2 was 100% in each sample. Based on existing reports identifying possible interactions of other keratin-family members with Annexin proteins, co-immunoprecipitation followed by western blot was done to confirm this finding. Immunoprecipitations were performed using antibody to annexin-A2, keratin-8, and non-immune IgG for negative control. Serial dilutions of the immunoprecipitation eluates were then probed by western blot with annexin-A2 and keratin-8 antibodies. Keratin-8 and annexin-A2 bands were present in each of the specific eluates and absent in the control IgG immunoprecipitation (FIG. 15).

Those skilled in the art will recognize, or be able to ascertain using no more than routine experimentation, many equivalents to the specific embodiments of the invention described herein. Such equivalents are intended to be encompassed by the following claims.

1. A method of treating a disease or disorder characterized by cells having increased or aberrant expression of cytokeratin-8 (CK8) comprising administering to a subject in need thereof a pharmaceutical composition comprising an effective amount of a CK8 inhibitor.

2. The method of claim 1, wherein the cells are cancer cells.

3. The method of claim 2, wherein the cancer cells are thyroid cancer cells.

4. The method of claim 3, wherein the thyroid cancer cells are poorly or undifferentiated.

5. The method of claim 4, wherein the thyroid cancer is anaplastic thyroid cancer or a poorly differentiated papillary thyroid cancer.

6. A method of treating cancer comprising administering to a subject with a cancer comprising cells having increased or aberrant expression of cytokeratin-8 (CK8) a pharmaceutical composition comprising an effective amount of a CK8 inhibitor.

7. The method of claim 6, wherein the cancer is a thyroid cancer.

8. The method of claim 7, wherein the thyroid cancer comprises poorly or undifferentiated cells

9. The method of claim 8, wherein the thyroid cancer is anaplastic thyroid cancer or a poorly differentiated papillary thyroid cancer.

10. A method of treating anaplastic thyroid cancer or a poorly differentiated papillary thyroid cancer in a subject comprising contacting anaplastic thyroid cancer or poorly differentiated papillary thyroid cancer cells of the subject a pharmaceutical composition comprising an effective amount of a CK8 inhibitor.

11. The method of claim 10, wherein the CK8 inhibitor reduces a bioactivity of CK8.

12. The method of claim 10, wherein the CK8 inhibitor reduces expression or localization of CK8 protein or mRNA.

13. The method of claim 10, wherein the CK8 inhibitor increases degradation of CK8 protein or mRNA.

14. The method of claim 10, wherein the CK8 inhibitor reduces proliferation of the cells.

15. The method of claim 10, wherein the CK8 inhibitor reduces cell-cell or cell-matrix adhesion of the cells.

16. The method of claim 10, wherein the CK8 inhibitor is a functional nucleic acid or one or more vectors encoding a functional nucleic acid, wherein the functional nucleic acid reduces expression of a nucleic acid comprising at least 80% sequence identity to SEQ ID NO:1, or a nucleic acid comprising at least 80% sequence identity to a polynucleotide encoding SEQ ID NO:2 or 4.

17. The method of claim 16, wherein the functional nucleic acid is selected from the group consisting of antisense oligonucleotides, siRNA, shRNA, miRNA, EGSs, ribozymes, and aptamers.

18. The method of claim 10, wherein the CK8 inhibitor is one or more vectors encoding a gene editing system that when transfected into the cells reduces, prevents or otherwise disrupts endogenous expression of CK8.

19. The method of claim 18, wherein the gene editing system is selected from the group consisting of CRISPR/Cas, zinc finger nucleases, and transcription activator-life effector nucleases.

20. The method of claim 10, wherein the CK8 inhibitor is an inhibitory anti-CK8 antibody or an antigen-binding fragment thereof.
